SEQUENCE LISTING (4 sequences)

SEQ ID NO: 1 - protein, 21 aa, Artificial Sequence
  alphas1(59-79) phosphopeptide
  MOD_RES (6)..(6): PHOSPHORYLATION
  MOD_RES (8)..(10): PHOSPHORYLATION
  MOD_RES (17)..(17): PHOSPHORYLATION
QMEAESISSSEEIVPNSVEQK

SEQ ID NO: 2 - protein, 25 aa, Artificial Sequence
  beta(1-25) phosphopeptide
  MOD_RES (15)..(15): PHOSPHORYLATION
  MOD_RES (17)..(19): PHOSPHORYLATION
RELEELNVPGEIVESLSSSEESITR

SEQ ID NO: 3 - protein, 25 aa, Artificial Sequence
  alphas2(46-70) phosphopeptide
  MOD_RES (11)..(13): PHOSPHORYLATION
  MOD_RES (16)..(16): PHOSPHORYLATION
NANEEEYSIGSSSEESAEVATEEVK

SEQ ID NO: 4 - protein, 21 aa, Artificial Sequence
  Alphas2(1-21) phosphopeptide
  MOD_RES (8)..(10): PHOSPHORYLATION
  MOD_RES (16)..(16): PHOSPHORYLATION
KNTMEHVSSSEESIISQETYK

(19) United States
(12) Patent Application Publication
(10) Pub. No.: US 2022/0183810 A1
(43) Pub. Date: Jun. 16, 2022

(54) CONCEALMENT OF HYPOMINERALISED LESIONS

(71) Applicant: The University of Melbourne, Victoria (AU)

(72) Inventor: Eric Charles Reynolds, Victoria (AU)

(73) Assignee: The University of Melbourne, Victoria (AU)

(21) Appl. No.: 17/438,299

(22) PCT Filed: Mar. 13, 2020
(86) PCT No.: PCT/AU2020/050239

§ 371 Date: Sep. 10, 2021

(30) Foreign Application Priority Data
Mar. 13, 2019 (AU) 2019900833
Oct. 14, 2019 (AU) 2019903860

(51) Int. Cl. A61C 19/06; A61K 6/20; A61K 6/838

(52) U.S. Cl. CPC A61C 19/063; A61K 6/838; A61K 6/20

(57) ABSTRACT

The present invention relates to compositions and uses for reducing the visibility of hypomineralised dental surfaces or subsurfaces, in particular in dental enamel. In one aspect, the invention involves a method of reducing visibility of a hypomineralised dental surface or subsurface, the method comprising (i) contacting the hypomineralised dental surface or subsurface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and (ii) subsequently to (i), raising the pH of the liquid composition applied to the hypomineralised dental surface or subsurface to equal to, or greater than, about 9, thereby forming a gel in and/or on the hypomineralised dental surface or subsurface, thereby reducing visibility of a hypomineralised dental surface or subsurface.

### CROSS-REFERENCE TO EARLIER APPLICATION(S)

This application claims priority to Australian provisional applications 2019900833 and 2019903860, the entire contents of each are herein incorporated by reference in their entirety.

### FIELD OF THE INVENTION

The present invention relates to compositions and uses for reducing the visibility of hypomineralised dental surfaces or subsurfaces, in particular in dental enamel.

### BACKGROUND OF THE INVENTION

Dental white spots are porous lesions in dental enamel/dentine which can result from dental caries (white spot lesions), hypomineralisation (e.g. fluorotic lesions and other developmental defects) or other demineralisation processes (e.g. erosion lesions). Typically they represent the early stage of caries formation where affected surfaces seem to be intact upon gentle probing. Other common causes of poor mineralization and associated white spot lesions include trauma, xerostomia, and arrested decay that has only partially remineralized around fixed orthodontic appliances, which may provide shelter for bacteria or interfere with normal remineralization.

This porous tissue in enamel and dentine has a different refractive index to that of translucent enamel and dentine hence the porous lesions appear as opaque white lesions against a translucent background. These lesions are not only a cosmetic concern but they also increase the risk of caries/erosion progression and hypersensitivity.

Current treatments of these lesions before cavitation involve remineralisation using fluorides with or without calcium phosphates but remineralisation is a slow process and it takes months to change the appearance of the lesions which often never completely disappear. Other treatments involve restoration which is invasive or at best micro-invasive involving infiltration with a methacrylate-based resin. These restorative processes are expensive, time consuming and do not provide a natural repair (remineralisation with calcium and phosphate) of the white spot which can cause later problems with discolouration of the restorative material or caries under the restoration due to a poor seal.

The best treatment to repair porous white spots and reduce sensitivity is to treat the lesions with CPP-ACP (available commercially as Recaldent™) with or without fluoride ions to effect remineralisation with hydroxyapatite or fluorohydroxyapatite.

However, current treatment methods with this technology still require many weeks of treatment at best to achieve an improvement in the appearance of the lesion and reduction in sensitivity. This can produce poor patient outcomes due to low compliance and inadequate remineralisation.

There is a need to provide new or improved methods for reducing the visibility of and/or forming a barrier to hypomineralised dental surfaces or subsurfaces.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

In one aspect, the present invention provides a method of reducing visibility of a hypomineralised dental surface or subsurface, the method comprising

contacting the hypomineralised dental surface or sub-surface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,

thereby reducing visibility of a hypomineralised dental surface or sub-surface.

Preferably, the pH of the liquid composition is greater than or equal to pH 6 but less than or equal to pH 8, for example greater than or equal to 7 but less than or equal to pH 8.

In a further aspect, the present invention provides a method of forming a protective layer on a dental surface, the method comprising:

contacting the dental surface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,

thereby forming a protective layer on the dental surface.

Preferably, the pH of the liquid composition is greater than or equal to pH 6 but less than or equal to pH 8, for example greater than or equal to 7 but less than or equal to pH 8.

In a further aspect, the present invention provides a method of treating or preventing dentinal sensitivity in a subject in need thereof, the method comprising:

contacting exposed dentinal tubules with contacting the dental surface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,

thereby treating or preventing dentinal sensitivity in the subject in need thereof.

Preferably, the pH of the liquid composition is greater than or equal to pH 6 but less than or equal to pH 8, for example greater than or equal to 7 but less than or equal to pH 8.

Preferably a heat source is applied to the liquid composition once it has contacted the dental surface or sub-surface.

In one aspect, the present invention provides a method of reducing visibility of a hypomineralised dental surface or subsurface, the method comprising:

(i) contacting the hypomineralised dental surface or subsurface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and

(ii) subsequently to (i), raising the pH of the liquid composition applied to the hypomineralised dental surface or subsurface to equal to, or greater than, about 9, thereby forming a gel in and/or on the hypomineralised dental surface or subsurface,

thereby reducing visibility of a hypomineralised dental surface or subsurface.

In a further aspect, the present invention provides a method of forming a gel in and/or on a dental surface or sub-surface lesion, the method comprising:

(i) contacting the dental surface or sub-surface lesion with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and

(ii) subsequently to (i), raising the pH of the liquid composition applied to the dental surface or sub-surface lesion to equal to, or greater than, about 9,

thereby forming a gel in and/or on the dental surface or sub-surface lesion.

In a further aspect, the present invention provides a method of reducing visibility of a hypomineralised dental surface or subsurface, the method comprising:

(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;

(ii) contacting the hypomineralised dental surface or subsurface with the mixed composition, thereby forming a gel in and/or on the hypomineralised dental surface or subsurface,

thereby reducing visibility of a hypomineralised dental surface or subsurface.

In a further aspect, the present invention provides a method of treating or preventing dentinal sensitivity in a subject in need thereof, the method comprising:

(i) contacting exposed dentinal tubules with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and

(ii) subsequently to (i), raising the pH of the liquid composition applied to the hypomineralised dental surface or subsurface to equal to, or greater than, about 9, thereby forming a gel in and/or on the exposed dentinal tubules,

thereby treating or preventing dentinal sensitivity in the subject in need thereof.

In a further aspect, the present invention provides a method of treating or preventing dentinal sensitivity in a subject in need thereof, the method comprising:

(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;

(ii) contacting exposed dentinal tubules with the mixed composition, thereby forming a gel in and/or on the exposed dentinal tubules,

thereby treating or preventing dentinal sensitivity in the subject in need thereof.

In a further aspect, the present invention provides a method of forming a protective layer on a dental surface, the method comprising:

(i) contacting the dental surface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and

(ii) subsequently to (i), raising the pH of the liquid composition applied to the dental surface to equal to, or greater than, about 9,

thereby forming a protective layer on the dental surface.

In a further aspect, the present invention provides a method of forming a protective layer on a dental surface, the method comprising:

(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;

(ii) contacting a dental surface with the mixed composition,

thereby forming a protective layer on the dental surface.

In any aspect of the present invention, the pH of the liquid composition applied to the dental surface or sub-surface lesion is raised to equal to, or greater than, about 9, preferably 10.

In any aspect of the present invention, the dental surface is preferably dental enamel. In one embodiment the dental surface is a lesion in the enamel, such as a lesion caused by caries, dental erosion or fluorosis. Typically, the lesion is a white spot lesion.

In any aspect of the present invention, the dental surface is dentine, for example exposed dentine. In one embodiment the exposed dentine is a tooth root, for example caused by recession.

In any aspect of the present invention, the dental surface is contacted with the mixed composition in less than 20, 15, 14, 13, 12, 11, 10, 9, 8, 7, 6, 5, 4, 3, 2 or 1 minutes after formation of the mixed composition.

In any aspect of the present invention where raising the pH of the liquid composition applied to the dental surface or sub-surface is required, this may be by contacting the liquid composition applied to the dental surface or sub-surface with a further composition of alkaline pH. The further composition may also be in liquid form and may be also be referred to herein as a further liquid composition. The alkaline pH of the further composition may be a pH of about 9, 10, 11, 12, 13 or 14. Preferably, the further composition has an alkaline pH of about 9 or higher.

In any aspect as described herein, the liquid composition may further comprise fluoride ions, preferably free fluoride ions. The fluoride ions may be present in the liquid composition at a concentration in the range of about 200 ppm to 50,000 ppm. In a preferred embodiment, the fluoride ions are at a concentration in the range of about 2,600 ppm to about 10,000 ppm. In a further preferred embodiment, the fluoride ions in the liquid composition are at a concentration of about 8,200 ppm, or about 6,500 ppm. The fluoride ions may be present in the liquid composition at a concentration of equal to or greater than any ppm described herein, particularly in the Examples. In another embodiment, the fluoride ions are at a concentration of about 5,200 ppm for 40% w/v CPP-ACP, about 5,850 ppm for 45% w/v CPP-ACP, about 6,500 ppm for 50% w/v CPP-ACP, about 8,200 ppm for 63% w/v CPP-ACP, or about 9,900 ppm for 75% CPP-ACP. In another embodiment, the fluoride ions are at a concentration of about 5,200 ppm for 40% w/w CPP-ACP or about 7,800 ppm for 60% w/w CPP-ACP.The fluoride ions may be from any suitable source. A source of fluoride ions may include free fluoride ions or fluoride salts. Examples of sources of fluoride ions include, but are not limited to the following: sodium fluoride, sodium monofluorophosphate, stannous fluoride, sodium silicofluoride, silver fluoride, silver diammine fluoride and amine fluoride. These may be provided in solution (typically an aqueous solution), or a suspension.

In any aspect of the invention, steps (i) and (ii), or any step whereby a liquid composition is applied to a dental surface or subsurface, may include or be followed by heating of the dental surface or subsurface, or lesion. In one embodiment, the method may comprise heating the hypomineralised dental surface or subsurface, or lesion, simultaneously or subsequently to contacting the hypomineralised dental surface or subsurface, or lesion, with a liquid composition comprising at least 40% w/v of phosphopeptide (PP)-stabilized ACP and/or ACFP at a pH of less than or equal to pH 6. In another embodiment, the method may comprise heating the hypomineralised dental surface or subsurface, or lesion, simultaneously or subsequently to raising the pH of the liquid composition applied to the hypomineralised dental surface or subsurface to equal to, or greater than, about 9. In another embodiment, the method may comprise heating the hypomineralised dental surface or subsurface, or lesion, simultaneously or subsequently to contacting the hypomineralised dental surface or subsurface, or lesion, with a liquid composition comprising at least 60% w/w of phosphopeptide (PP)-stabilized ACP and/or ACFP at a pH of greater than or equal to pH 5 but less than or equal to pH 9.

In any aspect of the invention, the method comprises heating the dental surface or subsurface, or lesion to a temperature greater than or equal to 40° C., greater than or equal to 45° C., greater than or equal to 50° C., greater than or equal to 55° C., greater than or equal to 60° C. or greater than or equal to 65° C.

In any aspect of the invention, the method comprises heating the dental surface or subsurface, or lesion to a temperature greater than 37° C. but equal to or less than 65° C., greater than 40° C. but equal to or less than 65° C., greater than 45° C. but equal to or less than 65° C., greater than 50° C. but equal to or less than 65° C., greater than 55° C. but equal to or less than 65° C., greater than 60° C. but equal to or less than 65° C.

In any aspect of the present invention, the liquid composition comprises greater than 40% w/v phosphopeptide (PP)-stabilized ACP and/or ACFP comprises greater than 45% w/v, greater than 50% w/v stabilized ACP and/or ACFP, greater than 55% w/v stabilized ACP and/or ACFP, greater than 60% w/v stabilized ACP and/or ACFP, greater than about 65% w/v stabilized ACP and/or ACFP, greater than about 70% w/v stabilized ACP and/or ACFP, or greater than about 75% w/v stabilized ACP and/or ACFP. In one embodiment the liquid composition comprises 63% w/v stabilized ACP and/or ACFP.

In any aspect of the present invention, the liquid composition comprises greater than 40% w/w phosphopeptide (PP)-stabilized ACP and/or ACFP comprises greater than 45% w/w, greater than 50% w/w stabilized ACP and/or ACFP, greater than 55% w/w stabilized ACP and/or ACFP, greater than 60% w/w stabilized ACP and/or ACFP, greater than about 65% w/w stabilized ACP and/or ACFP, greater than about 70% w/w stabilized ACP and/or ACFP, or greater than about 75% w/w stabilized ACP and/or ACFP.

In any aspect of the present invention, the liquid composition comprises greater than 40% w/v phosphopeptide (PP)-stabilized ACP and/or ACFP comprises greater than 40% w/v stabilized ACP and/or ACFP but less than 80% w/v stabilized ACP and/or ACFP, greater than 45% w/v stabilized ACP and/or ACFP but less than 80% w/v stabilized ACP and/or ACFP, greater than 50% w/v stabilized ACP and/or ACFP but less than 80% w/v stabilized ACP and/or ACFP, greater than 55% w/v stabilized ACP and/or ACFP but less than 80% w/v stabilized ACP and/or ACFP, greater than 60% w/v stabilized ACP and/or ACFP but less than 80% w/v stabilized ACP and/or ACFP, greater than 65% w/v stabilized ACP and/or ACFP but less than 80% w/v stabilized ACP and/or ACFP, greater than 70% w/v stabilized ACP and/or ACFP but less than 80% w/v stabilized ACP and/or ACFP, or greater than 75% w/v stabilized ACP and/or ACFP but less than 80% w/v stabilized ACP and/or ACFP.

In any aspect of the present invention, the liquid composition comprises greater than 40% w/w phosphopeptide (PP)-stabilized ACP and/or ACFP comprises greater than 40% w/w stabilized ACP and/or ACFP but less than 80% w/w stabilized ACP and/or ACFP, greater than 45% w/w stabilized ACP and/or ACFP but less than 80% w/w stabilized ACP and/or ACFP, greater than 50% w/w stabilized ACP and/or ACFP but less than 80% w/w stabilized ACP and/or ACFP, greater than 55% w/w stabilized

ACP and/or ACFP but less than 80% w/w stabilized ACP and/or ACFP, greater than 60% w/w stabilized ACP and/or ACFP but less than 80% w/w stabilized ACP and/or ACFP, greater than 65% w/w stabilized ACP and/or ACFP but less than 80% w/w stabilized ACP and/or ACFP, greater than 70% w/w stabilized ACP and/or ACFP but less than 80% w/w stabilized ACP and/or ACFP, or greater than 75% w/w stabilized ACP and/or ACFP but less than 80% w/w stabilized ACP and/or ACFP.

In any aspect of the invention, the hypomineralised dental surface or subsurface, or lesion, is contacted with the liquid composition in (i) for a period of time that allows the liquid composition to penetrate the dental surface, subsurface or lesion. In one embodiment, the hypomineralised dental surface or subsurface, or lesion, is contacted with the liquid composition in (i) for up to 20 minutes before raising the pH of the liquid composition applied to the hypomineralised dental surface or subsurface to equal to, or greater than, about 9. In one embodiment, the hypomineralised dental surface or subsurface, or lesion, is contacted with the liquid composition in (i) for at least about a few second to at least about 5 minutes, preferably at least about 5 minutes to about 20 minutes.

In any aspect of the invention, the method further comprises etching, preferably acid etching the hypomineralised surface, subsurface or lesion, prior to contacting with the liquid composition, for example in (i). The acid etching may be performed by any method known in the art. Typically, the acid etching may be by contacting the hypomineralised surface, subsurface or lesion with a composition comprising about 30% phosphoric acid or about 15% HCl. Preferably, a resin barrier is applied to protect soft tissue, e.g. a rubber dam or liquid rubber dam, before acid etching.

In any aspect, formation of the protective layer may be facilitated by a further step of heating as described herein and/or etching as described herein.

The stabilized ACP and/or ACFP as used herein is phosphopeptide stabilized. Preferably, the phosphopeptide (as defined below) is a casein phosphopeptide. Preferably, the ACP or ACFP is in the form of a casein phosphopeptide stabilized ACP or ACFP complex.

In a preferred embodiment, the phosphopeptide stabilized amorphous calcium phosphate (ACP) or amorphous calcium fluoride phosphate (ACFP) complex has tightly bound and loosely bound calcium, wherein the bound calcium in the complex is less than the tightly bound calcium in an ACP or ACFP complex formed at a pH of 7.0. Typically, the ACP or ACFP is predominantly in a basic form.

In a preferred embodiment, the calcium ion content of the stabilized ACP or ACFP complex is in the range of about 30 to 100 moles of calcium per mole of PP. More preferably, the calcium ion content is in the range of about 30 to about 50 moles of calcium per mole of PP.

In any aspect or embodiments as described herein, the stabilized ACP and/or ACFP may be in a formulation with additional calcium phosphate. Typically, the formulation includes a PP-stabilized ACP and/or ACFP complex together with at least an equal amount by weight of calcium phosphate.

In a preferred embodiment the PP-stabilized ACP and/or ACFP is in the form of a casein phosphopeptide stabilized ACP and/or ACFP complex.

Preferably, the phase of the ACP is predominantly a basic phase, wherein the ACP comprises predominantly the species Ca²⁺, PO₄³⁻ and OH⁻. The basic phase of ACP may have the general formula [Ca₃(PO₄)₂]ₓ[Ca₂(PO₄)(OH)] where x≥1. Preferably x=1-5. More preferably, x=1. Preferably the two components of the formula are present in equal proportions. Accordingly, in one embodiment, the basic phase of ACP has the formula Ca₃(PO₄)₂Ca₂(PO₄)(OH).

Preferably, the phase of the ACFP is predominantly a basic phase, wherein the ACFP comprises predominantly the species Ca²⁺, PO₄³⁻ and F⁻. The basic phase of

ACFP may have the general formula [Ca₃(PO₄)₂]ₓ[Ca₂(PO₄)F]_{y} where x≥1 when y=1 or where y≥1 when x=1. Preferably, y=1 and x=1-3. More preferably, y=1 and x=1. Preferably the two components of the formula are present in equal proportions. Accordingly, in one embodiment, the basic phase of ACFP has the formula Ca₃(PO₄)₂Ca₂(PO₄)F.

In one embodiment, the ACP complex consists essentially of phosphopeptides, calcium, phosphate and hydroxide ions and water.

In one embodiment, the ACFP complex consists essentially of phosphopeptides, calcium, phosphate, fluoride and hydroxide ions and water.

In a further aspect of the present invention there is provided a method for remineralizing a dental lesion, the method comprising:

(i) contacting the dental lesion with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and

(ii) subsequently to (i), raising the pH of the liquid composition applied to the dental lesion to equal to, or greater than, about 9, thereby forming a gel in and/or on the dental lesion,

thereby remineralizing the dental lesion.

In a further aspect of the present invention there is provided a method for remineralizing a dental lesion, the method comprising:

contacting the hypomineralised dental surface or subsurface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,

thereby remineralizing the dental lesion.

Preferably, the pH of the liquid composition is greater than or equal to pH 6 but less than or equal to pH 8, for example greater than or equal to 7 but less than or equal to pH 8.

Preferably the dental lesion is in enamel or dentine. Typically, the dental lesion is selected from the group consisting of one or more of a white spot lesion; a fluorotic lesion; a caries lesion; or a lesion caused by tooth erosion.

In any aspect or embodiment of the invention described herein, the liquid and further compositions, or the mixed composition, as described herein are applied to the mouth, tooth or lesion by the subject in need of treatment or by a dental health care professional.

In one embodiment, the dental surface is in need of such treatment. Therefore the invention includes in addition to the steps of any method described herein a step of identifying a subject suffering fluorosis, dental caries, dentinal hypersensitivity or dental calculus, a white spot lesion; a fluorotic lesion; a caries lesion; or a lesion caused by tooth erosion. Specifically, the dental surface in need of the formation of a protective layer may be exposed dentine, typically causing dentinal hypersensitivity in a subject. The exposed dentine may have exposed dentinal tubules.

Typically the tooth surface may be one that has been identified as benefiting from a surface layer, for example, due to an increased likelihood of demineralization.

The methods of the invention that form a protective layer on a dental surface find particular application in occluding exposed dentine, particularly exposed dentine that has exposed dentinal tubules.

The dental surface may also be a cavity, whereby a method of the invention described herein is applied to the cavity thereby allowing formation of a gel or protective layer. A dental restorative such as a composite or glass ionomer cement can then be added on to the gel or protective layer.

In a further aspect, the present invention provides a method of restoring a dental cavity, the method comprising

(i) forming a gel or protective layer in the cavity by any method described herein; and

(ii) applying a dental restorative,

thereby restoring the dental cavity.

The present invention provides a liquid composition comprising at least 40% w/v of phosphopeptide (PP)-stabilized amorphous calcium phosphate (ACP) and/or amorphous calcium fluoride phosphate (ACFP) at a pH of less than or equal to pH 6 and a further composition of alkaline pH for use in mineralizing a dental surface or sub-surface, wherein the further composition is applied to the dental surface or sub-surface after the first composition thereby forming a gel.

The present invention provides a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9 for use in

forming a layer on a dental surface,

forming a gel in and/or on a dental surface or subsurface lesion,

treating or preventing dentinal hypersensitivity,

reducing the visibility of a hypomineralized dental surface or subsurface, or remineralizing a dental surface or subsurface.

Preferably, the pH of the liquid composition is greater than or equal to pH 6 but less than or equal to pH 8, for example greater than or equal to 7 but less than or equal to pH 8.

In a further aspect, there is provided a method of treating or preventing one or more of each of dental caries, tooth decay, dental erosion, fluorosis and white spot lesions, comprising the steps of

(i) contacting the dental surface or subsurface with a liquid composition comprising at least 40% by weight of phosphopeptide (PP)-stabilized amorphous calcium phosphate (ACP) and/or amorphous calcium fluoride phosphate (ACFP) at a pH of less than or equal to pH 6;

(ii) subsequently to (i), raising the pH of the liquid composition applied to the dental lesion to equal to, or greater than, about 9, thereby causing the first composition to form a gel.

Topical administration of the compositions is preferred. The method preferably includes the administration of the complex in a formulation as described above.

In a further aspect, there is provided a use of a phosphopeptide (PP)-stabilized amorphous calcium phosphate (ACP) and/or amorphous calcium fluoride phosphate (ACFP) in the manufacture of a product comprising a liquid composition for reducing visibility of a hypomineralised dental surface or subsurface, the liquid composition comprising at least 40% w/v of said phosphopeptide (PP)-stabilized amorphous calcium phosphate (ACP) and/or amorphous calcium fluoride phosphate (ACFP) at a pH of less than or equal to pH 6,

wherein the liquid composition is applied to the hypomineralised dental surface or subsurface and subsequently the pH of the liquid composition applied to the hypomineralised dental surface or subsurface is raised to equal to, or greater than, about 9, thereby forming a gel in and/or on the hypomineralised dental surface or subsurface.

In a further aspect, there is provided a use of a phosphopeptide (PP)-stabilized amorphous calcium phosphate (ACP) and/or amorphous calcium fluoride phosphate (ACFP) in the manufacture of a product comprising a liquid composition for forming a gel in and/or on a dental surface or subsurface lesion, the liquid composition comprising at least 40% w/v of said phosphopeptide (PP)-stabilized amorphous calcium phosphate

(ACP) and/or amorphous calcium fluoride phosphate (ACFP) at a pH of less than or equal to pH 6,

wherein the liquid composition is applied to the dental surface or subsurface lesion and subsequently the pH of the liquid composition applied to the dental surface or subsurface lesion is raised to equal to, or greater than, about 9, thereby forming a gel in and/or on the dental surface or subsurface lesion.

In a further aspect, there is provided a use of a phosphopeptide (PP)-stabilized amorphous calcium phosphate (ACP) and/or amorphous calcium fluoride phosphate (ACFP) in the manufacture of a product comprising or consisting of a liquid composition for:

forming a layer on a dental surface,

forming a gel in and/or on a dental surface or subsurface lesion,

treating or preventing dentinal hypersensitivity,

reducing the visibility of a hypomineralized dental surface or subsurface, or

remineralizing a dental surface or subsurface,

the liquid composition comprising at least 40% w/w of said phosphopeptide (PP)-stabilized amorphous calcium phosphate (ACP) and/or amorphous calcium fluoride phosphate (ACFP) at a pH of greater than or equal to pH 5 but less than or equal to pH 9. In one embodiment, the product is a cosmetic product.

In a further aspect, there is provided a use of a base in the manufacture of a composition for reducing visibility of a hypomineralised dental surface or subsurface in a subject who has received a liquid composition comprising at least 40% w/v of a phosphopeptide (PP)-stabilized amorphous calcium phosphate (ACP) and/or amorphous calcium fluoride phosphate (ACFP) at a pH of less than or equal to pH 6 to a hypomineralised dental surface or subsurface.

In a further aspect, there is provided a use of a base in the manufacture of a composition for forming a gel in and/or on a dental surface or subsurface lesion in a subject who has received a liquid composition comprising at least 40% w/v of a phosphopeptide (PP)-stabilized amorphous calcium phosphate (ACP) and/or amorphous calcium fluoride phosphate (ACFP) at a pH of less than or equal to pH 6 to a dental surface or subsurface lesion.

In a further aspect, there is provided a use of a phosphopeptide (PP)-stabilized amorphous calcium phosphate (ACP) and/or amorphous calcium fluoride phosphate

(ACFP) and a base in the manufacture of a product comprising a liquid composition and a further composition, the liquid composition comprising at least 40% w/v of said phosphopeptide (PP)-stabilized amorphous calcium phosphate (ACP) and/or amorphous calcium fluoride phosphate (ACFP) at a pH of less than or equal to pH 6, the further composition comprising said base, and the liquid composition and further composition being used to reducing visibility of a hypomineralised dental surface or subsurface, wherein the further composition is applied to the hypomineralised dental surface or subsurface after the liquid composition raising the pH of the liquid composition applied to the hypomineralised dental surface or subsurface to equal to, or greater than, about 9, thereby forming a gel in and/or on the hypomineralised dental surface or subsurface, thereby reducing visibility of a hypomineralised dental surface or subsurface. In one embodiment, the product is a cosmetic product.

In a further aspect, there is provided a use of a phosphopeptide (PP)-stabilized amorphous calcium phosphate (ACP) and/or amorphous calcium fluoride phosphate (ACFP) and a base in the manufacture of a product comprising a liquid composition and a further composition, the liquid composition comprising at least 40% w/v of said phosphopeptide (PP)-stabilized amorphous calcium phosphate (ACP) and/or amorphous calcium fluoride phosphate (ACFP) at a pH of less than or equal to pH 6, the further composition comprising said base, and the liquid composition and further composition being used to forming a gel in and/or on a dental surface or subsurface lesion, wherein the further composition is applied to the dental surface or subsurface lesion after the liquid composition raising the pH of the liquid composition applied to the dental surface or subsurface lesion to equal to, or greater than, about 9, thereby forming a gel in and/or on the dental surface or subsurface lesion.

In a preferred embodiment, the phosphopeptide stabilized amorphous calcium phosphate (ACP) or amorphous calcium fluoride phosphate (ACFP) complex in the composition has tightly bound and loosely bound calcium, wherein the bound calcium in the complex is less than the tightly bound calcium in an ACP or ACFP complex formed at a pH of 7.0. Optionally, the ACP or ACFP is predominantly in a basic form.

In another preferred embodiment, the calcium ion content of the stabilized ACP or ACFP complex in the composition is in the range of about 30 to 100 moles of calcium per mole of PP. More preferably, the calcium ion content is in the range of about 30 to about 50 moles of calcium per mole of PP.

In any embodiment, the ACP and/or ACFP in the composition can be in the form of a casein phosphopeptide stabilized ACP and/or ACFP complex.

In another aspect, the present invention provides a liquid composition comprises at least 40% w/v stabilized ACP and/or ACFP. Preferably, the liquid composition comprises greater than or equal to 45% w/v w/v stabilized ACP and/or ACFP, greater than or equal to 50% w/v stabilized ACP and/or ACFP, greater than or equal to 55% w/v stabilized ACP and/or ACFP, greater than or equal to 60% w/v stabilized ACP and/or ACFP, greater than or equal to 65% w/v stabilized ACP and/or ACFP, greater than or equal to 70% w/v stabilized ACP and/or ACFP, or greater than or equal to 75% w/v stabilized ACP and/or ACFP. In one embodiment the liquid composition comprises 63% w/v stabilized ACP and/or ACFP. Preferably, the liquid composition further comprises fluoride ions as described herein.

In this aspect of the present invention, the liquid composition may comprise greater than 40% w/v stabilized ACP and/or ACFP but less than 80% w/v stabilized ACP and/or ACFP, greater than 45% w/v stabilized ACP and/or ACFP but less than 80% w/v stabilized ACP and/or ACFP, greater than 50% w/v stabilized ACP and/or ACFP but less than 80% w/v stabilized ACP and/or ACFP, greater than 55% w/v stabilized ACP and/or ACFP but less than 80% w/v stabilized ACP and/or ACFP, greater than 60% w/v stabilized ACP and/or ACFP but less than 80% w/v stabilized ACP and/or ACFP, greater than 65% w/v stabilized ACP and/or ACFP but less than 80% w/v stabilized ACP and/or ACFP, greater than 70% w/v stabilized ACP and/or ACFP but less than 80% w/v stabilized ACP and/or ACFP, or greater than 75% w/v stabilized ACP and/or ACFP but less than 80% w/v stabilized ACP and/or ACFP. Preferably, the liquid composition further comprises fluoride ions as described herein.

In any aspect, the liquid composition is degassed. Degassing may be by any method that forms a negative pressure above the liquid composition. Exemplary methods involve a vacuum pump or system, for example a venturi vacuum water system.

Any composition described herein can be used in any one of the methods described herein. The composition is a physiologically acceptable composition as described herein.

In any method or use of the invention described herein, the method or use is for the cosmetic purpose of masking or concealing the visual appearance of a hypomineralised surface or subsurface.

In another aspect, the present invention provides a method or process for preparing a liquid composition comprising at least 40% w/v PP stabilized ACP and/or ACFP, the method or process comprising or consisting of:

mixing a solvent and a powder comprising or consisting of PP stabilized-ACP and/or ACFP, and

maintaining the pH below 7. Preferably, the pH is maintained at, or below, 6, preferably the pH is maintained at, or below, 5.5.

In another aspect, the present invention provides a method or process for preparing a liquid composition comprising at least 40% w/v PP stabilized ACP and/or ACFP, the method or process comprising or consisting of:

mixing a solvent and a powder comprising or consisting of PP stabilized ACP and/or ACFP, and

lowering the pH below 7. Preferably, the pH is lowered to, or below, 6, preferably 5.5. Typically, the pH is maintained below, 7, more preferably the pH is maintained at, or below, 6, even more preferably 5.5.

In any aspect, the step of mixing a solvent and a powder comprising or consisting of PP stabilized ACP and/or ACFP, comprises adding the solvent to the powder. Alternatively, the step comprises adding the powder to the solvent.

In any method or process for preparing a liquid composition as described herein, the method or process further comprises the step of degassing the liquid composition. Degassing may be by any method that forms a negative pressure above the liquid composition, including methods described herein.

In any method or process for preparing a liquid composition as described herein, the method or process further comprises a step of mixing the liquid composition with a solution comprising fluoride ions.

In another aspect, the present invention provides a method or process for preparing a liquid composition comprising at least 40% w/v PP stabilized ACP and/or ACFP, the method or process comprising or consisting of the steps as described in Example 1 herein.

In any aspect, the present invention provides a method or process that further comprises the following steps to prepare a powder comprising or consisting of PP stabilized-ACP and/or ACFP:

admixing one or more solutions comprising phosphopeptides, calcium ions, phosphate ions, hydroxide ions and optionally fluoride ions, while maintaining the pH at about 7.0 or above, preferably about 9, to form a solution comprising stabilized-ACP and/or ACFP, and

drying the solution comprising PP stabilized-ACP and/or ACFP,

thereby forming a powder comprising or consisting of PP stabilized-ACP and/or ACFP. Preferably drying is spray drying or freeze drying.

In one embodiment, the method or process further comprises the steps;

filtering the solution comprising PP stabilized-ACP and/or ACFP, prior to drying, to form a retentate, wherein the retentate is subsequently dried to form powder comprising or consisting of PP stabilized-ACP and/or ACFP.

In another aspect, the present invention provides a method or process for preparing a liquid composition comprising at least 40% w/w PP stabilized ACP and/or ACFP, the method or process comprising or consisting of:

mixing a solvent and a powder comprising or consisting of PP stabilized ACP and/or ACFP, and

lowering the pH below 8. Preferably the solvent comprises fluoride.

In this aspect, the method further comprises a step of stirring the liquid composition after the pH is lowered. Preferably, the stirring occurs for at least 5, 10, 15, 20, 25 or 30 minutes.

In this aspect, the liquid composition is degassed to remove trapped air bubbles, preferably by placing the solution under vacuum, most preferably for 24 hours.

In another aspect, the present invention provides a method or process for preparing a liquid composition comprising at least 40% w/w, preferably 60% w/w, PP stabilized ACP and/or ACFP, the method or process comprising or consisting of the steps as described in Example 11 herein.

In any method or process for preparing a liquid composition comprising greater than 40% w/v or 40% w/w stabilized ACP and/or ACFP, the solvent is water.

In any method or process for preparing a liquid composition comprising greater than 40% w/v or 40% w/w stabilized ACP and/or ACFP, the pH is lowered or maintained using 1-10 M HCl, or 11M HCl.

In any aspect, the method or process for preparing a liquid composition comprising at least 40% w/v PP stabilized ACP and/or ACFP, may be for preparing a liquid composition comprising equal to or greater than 45%, 50%, 55%, 60%, 65%, 70%, or 75% w/v (or any other % w/v described herein) stabilized ACP and/or ACFP.

In any method or process for preparing a liquid composition comprising at least 40% w/v PP stabilized ACP and/or ACFP, the PP stabilized ACP or ACFP is CPP-ACP or CPP-ACFP as described herein.

In any method or process for preparing a liquid composition comprising at least 40% w/v PP stabilized ACP and/or ACFP, the liquid composition is for use in any method of dental treatment, preferably those described herein (e.g. reducing visibility of a hypomineralised dental surface or subsurface).

In another aspect, the present invention provides a liquid composition comprising at least 40% w/v PP stabilized ACP and/or ACFP prepared by a method or process described herein.

The invention also relates to a kit for the treatment or prevention of one or more of dental caries, fluorosis, dental erosion and white spot lesions including (a) a liquid composition comprising at least 40% by weight of phosphopeptide (PP)-stabilized amorphous calcium phosphate (ACP) and/or amorphous calcium fluoride phosphate (ACFP) at a pH of less than or equal to pH 6 and (b) a further composition of alkaline pH, preferably as described herein, wherein the further composition is applied to the dental surface or sub-surface after the liquid composition and wherein the further (or second) composition causes the liquid (or first) composition to gel. Desirably, the kit further includes instructions for their use for the mineralization of a dental surface in a patient in need of such treatment, preferably for use in any method described herein.

The instructions may describe the use of the kit to treat or prevent one or more of each of dental caries, tooth decay, dental erosion, fluorosis and white spot lesions. In one embodiment, the liquid (or first) composition and the further (or second) composition are present in suitable amounts for treatment of a patient. Preferably, the phosphopeptide (as defined below) is a casein phosphopeptide. Preferably, the PP-stabilized ACP or ACFP is in the form of a casein phosphopeptide stabilized ACP or ACFP complex.

In one embodiment the present invention provides any kit as described herein for use, or when used, in a cosmetic method of masking or concealing the visual appearance of a hypomineralised surface or subsurface. Typically, the kit may further comprises written instructions for use in a cosmetic method as described herein.

The kit of the invention may further include a source of free fluoride ions. Examples of sources of free fluoride ions include, but are not limited to the following: sodium fluoride, stannous fluoride, silver fluoride, amine fluoride or any metal ion fluoride salt. These source of fluoride ions may be provided in solution (typically an aqueous solution), or a suspension.

In one aspect, the present invention also provides a kit comprising or consisting of:

(a) a first composition comprising a powder of phosphopeptide stabilized ACP and/or ACFP;

(b) a second composition comprising a solution of fluoride at a pH of less than or equal to pH 6; and

(c) a third composition of alkaline pH.

Preferably, the alkaline pH of the third composition may be a pH of about 9, 10, 11, 12, 13 or 14. Preferably, the third composition has an alkaline pH of about 9 or higher. Preferably, the first composition comprises an amount of phosphopeptide stabilized ACP and/or ACFP that when mixed with the second composition, a liquid composition comprising at least 40% w/v of phosphopeptide stabilized ACP and/or ACFP is formed.

In one embodiment, the kit comprises or consists of:

(a) 5 g of CPP-ACP and/or CPP-ACFP,

(b) 5 ml of 0.73 M NaF in 1.146 M HCl, and

(c) 1.5 M NaOH.

Preferably, the kit further includes two microbrushes.

Preferably, the kit further comprises written instructions to use the kit in any method described herein.

### BRIEF DESCRIPTION OF THE DRAWINGS

FIG. 1. Masking of white spot lesion using a 45% w/v CPP-ACP pH 5.5 solution followed by a 1M NaOH solution. Treated lesion on the left, control on the right.

FIG. 2. Masking of white spot lesion using a 63% w/v CPP-ACP and 8,200 ppm F as NaF pH 5.5 solution (not degassed) followed by a 1M NaOH solution. Treated lesion on the left, control on the right.

FIG. 3. Using the dental kit described in Example 3, part (a) was added to part (b) with thorough mixing. This mixture was then applied with one microbrush to the white spot lesions on the left (see image in (A) showing lesions before application). After a few secs/mins at 37° C. solution (c) was then applied to the white spot lesions on the left with the second microbrush. Within 15 mins the reaction in the white spot occurred to form a gel and concealed the white spots (see image in (B)).

FIG. 4. Masking of white spot lesion using a 75% w/v CPP-ACP and 9,880 ppm F as NaF pH 5.5 solution followed by a 2 M NaOH solution. Treated lesion on the left, control on the right.

FIG. 5. Masking of white spot lesion using a mixed composition formed from a liquid composition of 63% w/v CPP-ACP and 8,000 ppm F pH 5.5 (degassed) and a solution of 1.5 M NaOH. Treated lesion on the left, control on the right.

FIG. 6. Treatment of demineralised dentine using a mixed composition formed from a liquid composition of 63% w/v CPP-ACP and 8,000 ppm F pH 5.5 and a solution of 1.5 M NaOH. Treated lesion on the left, control on the right.

FIG. 7. (A and B) Representative transverse microradiographic image (TMR) image showing formation of a protective layer over demineralised dentine using a mixed composition formed from a liquid composition of 63% w/v CPP-ACP and 8,000 ppm F pH 5.5 and a solution of 1.5M NaOH. This is a TMR image taken about 20 min after applying the mixed composition.

FIG. 8. Representative scanning electron microscopy (SEM) image showing formation of a protective layer over demineralised dentine in (A) using a mixed composition formed from a liquid composition of 63% w/v CPP-ACP and 8,000 ppm F pH 5.5 and a solution of 1.5 M NaOH, and control dentine with exposed dentinal tubules in (B).

FIG. 9. Representative scanning electron microscopy (SEM) image showing deliberate cracking upon dehydration of the protective layer from FIG. 8 in (A) and elemental analysis by SEM-EDS of the protective layer in (B).

FIG. 10. Microradiographed image showing the formation of a protective layer over root dentine treated with premixed CPP-ACFP and 2 M NaOH, incubated at 37° C. for 48 hours.

FIG. 11. Microradiographed image showing the formation of a protective layer over etched enamel surface treated with premixed CPP-ACFP and 2 M NaOH, incubated at 37° C. for 48 hours.

FIG. 12. Masking of white spot lesion using a 60% w/w CPP-ACP with 7,800 ppm F at pH 7.8 (75% w/v CPP-ACP containing 10,000 mg/L F at pH 7.8) composition described in Example 11. (a) Prior to treatment, (b) after 40 seconds of curing, (c) 15 minutes after curing, and (d) 23 hours after curing.

### DETAILED DESCRIPTION OF THE INVENTION

Further aspects of the present invention and further embodiments of the aspects described in the preceding paragraphs will become apparent from the following description, given by way of example and with reference to the accompanying drawings. It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps. As used herein, except where the context requires otherwise, “comprise” and “include” can be used interchangeably.

An aspect of the current invention is based on several surprising findings, the first that a composition comprising a high concentration of phosphopeptide (PP)-stabilized amorphous calcium phosphate (ACP) and/or amorphous calcium fluoride phosphate (ACFP) can remain in a liquid state (i.e. does not form a gel). Prior to the present invention it was thought that high concentrations of phosphopeptide-stabilized ACP and/or ACFP would result in the composition forming a gel or paste, and all liquid compositions described to date had relatively low concentrations of phosphopeptide-stabilized ACP and/or ACFP. The second surprising finding is that raising the pH of the acidic liquid composition changes the form of the composition to a gel, and this can occur in and/or on a dental surface, subsurface or lesion. Without being bound by any theory or mode of action it is believed that raising the pH destabilises the PP-ACP and/or PP-ACFP complexes to form an amorphous embryonic hydroxyapatite or fluorohydroxyapatite gel. The third surprising finding is that this formation of a gel occurs rapidly after raising the pH of the composition, for example by applying a further composition with an alkaline pH. The fourth surprising finding is that the gel that is formed changes the refractive index and reduces the visibility of the hypomineralised surface, subsurface or lesion by returning the hypomineralised surface, subsurface or lesion to translucency. An advantage of the present invention is that it masks or conceals visible hypomineralised lesions to substantially improve the appearance and reduce visibility, i.e. to return the surface to translucency, within minutes in a dental surgery during a single patient visit. Further, it does so with calcium and phosphate (with or without fluoride). This represents a substantial improvement in current in-surgery dental treatments of, for example, white spot lesions. At the same time as providing a cosmetic benefit, the gel then provides a reservoir of high concentrations of calcium, phosphate and optionally fluoride, to remineralize the surface, subsurface or lesion.

The liquid composition comprising a high concentration of phosphopeptide-stabilized ACP and/or ACFP is typically at a pH of 6 or less can be combined or mixed with a further liquid composition of alkaline pH prior to application to the dental surface. A further surprising finding is that the combined or mixed composition formed, which comprises a high concentration of phosphopeptide-stabilized ACP and/or ACFP where the pH is equal to, or greater than, about 9, is maintained in a state that allows it to be applied to a dental surface. In other words, the combined or mixed composition exists in a liquid form for a time, for example 1 to 2 minutes, which allows application to a dental surface.

A yet another surprising finding is that a composition comprising a high concentration of phosphopeptide (PP)-stabilized amorphous calcium phosphate (ACP) and/or amorphous calcium fluoride phosphate (ACFP) can remain in a liquid state (i.e.

does not form a gel) and does so at a near neutral pH. This composition does not need to be prepared at the dental professional surgery, at the physical location where the individual is treated. This liquid composition can then be used to reduce the visibility of white spot lesions, reduce dentinal hypersensitivity and other uses as described herein.

A dental subsurface is typically a hypomineralised lesion such that the first composition and the second composition, or mixed composition, contacted to the dental surface migrates through any surface layer, i.e. pellicle and/or plaque, through the porous dental surface to the region requiring mineralization. Preferably, the PP-stabilized ACP or ACFP is in the form of a casein phosphopeptide stabilized ACP or ACFP complex. The dental surface is preferably dental enamel. The dental surface may be a lesion in the enamel, such as a lesion caused by caries, dental erosion or fluorosis.

A reduction in visibility of a hypomineralised surface, subsurface or lesion can be determined simply by visual inspection by the human eye. A reduction in visibility may be any level of reduction such that the hypomineralised surface, subsurface or lesion is less noticeable. A reduction in visibility may result in the hypomineralised surface, subsurface or lesion adopting a translucent appearance such that there is little or no difference with surrounding normal, mineralised dental surface as determined by the human eye.

Visibility of a surface, subsurface or lesion may also be determined as follows. A Chroma Meter (Minolta ChromaMeter CR241, Minolta, Japan) can be used to record surface reflectance. Surface reflectance measurement was established in L*a*b* color space by the Commission de L'Eclairage in 1978, and measurements relate to human colour perception in three colour dimensions (Commision Internationale de L'Eclaige (1978). Recommendations on uniform colour spaces, colour difference equations and psychometric colour terms. Paris: Bureau Centrale de la DIE Suppl. 2:15.). The L* values represent colour gradients from white to black, a* values represent colour gradients from green to red, and b* values represent colour gradients from blue to yellow (Commision Internationale de L'Eclaige, 1978). Only L* value measurements may be used with whiter colours having a higher reading, and darker colours a lower reading. To ensure a reproducible position of specimens in the Chroma Meter, a wax mold for each sample may be prepared and stored. All samples may be air-dried with a dental triplex syringe for 60 s before each measurement. Individual specimens may be repositioned ten times both before and after treatment, and colour reflectance L* values were recorded.

Dentinal hypersensitivity results when protective enamel or cementum covering dentine is lost. Cementum is typically easier to breach than enamel, because cementum is thinner and more easily eroded by acids. However, breach of cementum cannot happen until there is gingival recession and exposure of the root surface to the oral environment. Individuals with breached cementum and suffering with dentinal hypersensitivity often experience pain when the exposed area of the tooth comes into contact with cold air, hot and cold liquids, foods that are sweet or acidic, or is touched with a metal object. Patients suffering from tooth hypersensitivity have larger number of open dentinal tubules and/or tubules with a larger diameter than normal.

An advantage of an aspect of the present invention is the formation of a protective layer. This layer typically has the same, or similar, composition as hydroxyapatite or fluorapatite. It can form on enamel or dentine and can be used to seal or occlude dentinal tubules thereby reducing dentinal sensitivity.

Such a layer may be characterised has a calcium : phosphate ratio equivalent to normal apatite, preferably where the ratio is about 1.5-2:1. The layer ideally contains an amount of calcium that is about 20 wt %.

Preferably, the layer contains carbon, oxygen, phosphate and calcium, and optionally fluoride.

Methods of the invention that result in sealing of exposed dentine reduce tooth sensitivity and reduce the risk of caries, for example tooth root surface caries. Further, as dental restoratives can shrink and form microgaps with the dental surface, the present invention would find particular application prior to applying a restorative material such as a composition that include glass ionomer cement. The gel or protective layer would then act as a cavity sealer and reduce the formation of microgaps.

The words ‘treat’ or ‘treatment’ refer to therapeutic treatment wherein the object is to slow down (lessen) an undesired physiological change or disorder. For the purposes of this invention, beneficial or desired clinical results include, but are not limited to, alleviation of symptoms, diminishment of extent of the condition, stabilized (i.e., not worsening) state of the condition, delay or slowing of condition progression, amelioration or palliation of the disease/condition state, and remission (whether partial or total), whether detectable or undetectable. Treatment may not necessarily result in the complete absence of detectable symptoms of the condition but may reduce or minimise complications and side effects of the condition. The success or otherwise of treatment may be monitored by physical examination of the individual or response to any thermal, tactile or chemical treatment as described herein. Where a method of the invention is used to treat a subject for dentinal sensitivity, or hypersensitivity, preferably, the subject experiences a reduction in the severity of the pain or a reduction in the incidence of pain over time. Methods for identifying subjects having different degrees of dentinal sensitivity, and for measuring success of treatment or prevention, are described herein and also include those outlined in Med Oral Patol Oral Cir Bucal. 2008 Mar. 1; 13(3): E201-6. Treatment of a subject may be determined by comparing the level of pain experienced when exposed to any stimuli described herein before and after treatment, whereby a reduction in pain after treatment indicates a reduction in sensitivity.

The words ‘prevent’ and ‘prevention’ generally refer to prophylactic or preventative measures for protecting or precluding an individual not having a condition or symptom, for example sensitivity, from progressing to having the condition or symptom, for example sensitivity. Individuals in whom prevention may be required are those undergoing a dental procedure, particularly a dental procedure that exposes dentine.

In any aspect of the invention, the hypomineralised dental surface or subsurface, or lesion, is contacted with the liquid composition in (i) for a period of time that allows the liquid composition to penetrate the dental surface, subsurface or lesion. Typically, the liquid composition is applied for a period of time that allows the liquid composition to penetrate porosities of the hypomineralised dental surface or subsurface, or lesion. This then provides the liquid composition within those porosities so that the visibility of the the hypomineralised dental surface or subsurface, or lesion can be reduced when the pH of the liquid composition in the porosities is raised thereby forming a gel. The gel may therefore be formed in the subsurface or lesion, or in and on the subsurface or lesion. In one embodiment, the hypomineralised dental surface or subsurface, or lesion, is contacted with the liquid composition in (i) for up to 20 minutes before raising the pH of the liquid composition applied to the hypomineralised dental surface or subsurface to equal to, or greater than, about 9. In one embodiment, the hypomineralised dental surface or subsurface, or lesion, is contacted with the liquid composition in (i) for at least about a few second to at about 5 minutes, preferably at least about 5 minutes to about 20 minutes.

Typically the gel is formed any time from when the further composition is applied until about 5 to 20 minutes. Therefore, in one embodiment the further composition is applied to raise the pH of the first composition and a period of about 5 to about 20 minutes is allowed to pass before any further compositions are applied to, or procedures conducted on, the dental surface, subsurface or lesion.

As used herein % w/v may be taken to be equivalent to g/100 ml.

In any aspect the dental surface is in need of such treatment. Therefore, in another aspect, the invention includes in addition to the steps of any method described herein a step of identifying a subject suffering fluorosis, dental caries, dentinal hypersensitivity or dental calculus, a white spot lesion; a fluorotic lesion; a caries lesion; or a lesion caused by tooth erosion.

A further composition of alkaline pH is a composition with a pH greater than 7 that includes a base or a compound capable of producing a base. A base is defined as a compound which can accept hydrogen cations (protons) or, more generally, donate a pair of valence electrons. The composition may include a compound that may not necessarily normally be regarded as a base, for example a polypeptide with numerous acidic and basic residues but nonetheless has the ability to increase the pH of the composition to greater than 7, preferably to pH about 9 or greater. Non-limiting examples of bases suitable for use in the invention include hydroxides, borates, phosphates including hydrogen phosphates, amines and any salt forms thereof including an alkali metal salt forms. More specifically, non-limiting examples of suitable pharmaceutically acceptable bases include ammonium hydroxide, sodium hydroxide, potassium hydroxide, lithium hydroxide, calcium hydroxide, magnesium hydroxide, ferrous hydroxide, zinc hydroxide, sodium hypochlorite, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine or urea. In one embodiment, the further composition comprises sodium hydroxide, preferable at a concentration of greater than or equal to 1 or 2 M.

Any pharmaceutically acceptable compounds described as a base are suitable for use in the invention. Typically, the base is suitable for oral use. Preferably, the compound acts as a base, i.e. only releases hydroxide ions or donates electrons, in the presence of an acid. The base may be a free-base form, or in a pharmaceutically acceptable salt form. Non-limiting examples of bases suitable for use in the invention include hydroxides, borates, phosphates including hydrogen phosphates and dihydrogen phosphates, citrates, carbonates, bicarbonates, hypochlorites, amines and any salt forms thereof including an alkali metal salt forms. More specifically, non-limiting examples of suitable pharmaceutically acceptable bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine. A hypofluorite capable of acting as a base as described herein is also useful in the invention as the agent for increasing or maintaining pH. A suitable hypofluorite would react in situ to produce fluoride ions and hydroxide (or another base) ions. One skilled in the art will appreciate that fluoride ions can substitute for hydroxide in the crystal structure of apatite forming fluorapatite.

Any heat source may be used in a method or use of the invention to heat or cure the dental surface or subsurface. Heat sources that emit light or radiation and are suitable for use in dental applications are known in the art. Specific examples include dental curing lights, for example a 10W high-power blue light LED such as X-Cure by Guilin Woodpecker Medical Instrument Co. Ltd. In any method or use of the invention, there may be an additional step of heating or curing the liquid composition once it has contacted the dental surface or sub-surface. The heating or curing may be for a period of equal to or at least 30 seconds, equal to or at least 40 seconds, equal to or at least 50 seconds, equal to or at least 60 second, equal to or at least 2 minutes or equal to or at least 5 minutes. The heating or curing may increase temperature in bursts to 45-50° C. (with patient comfort). The heading or curing may be for any time or at any temperature as described herein including the examples.

Any composition described herein may be applied to a dental surface, subsurface or lesion using any technique known in the art or described herein. An exemplary application technique is using a microbrush.

Further, any resin barrier, such as a rubber dam, may be used to protect soft tissue in the oral cavity from application of any composition described herein.

As used herein, “stabilized-ACP or ACFP” and “stabilized-ACP or ACFP complex” are used interchangeably.

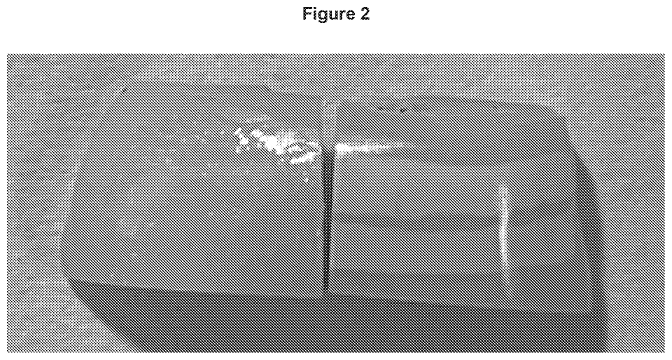

A stabilized-ACP or ACFP complex as described in the current specification may be the “closed” complexes as shown in FIG. 2 of Cross et al., 2007.

A stabilized-ACP or ACFP complex as referred to herein includes a stabilized-ACP or ACFP complex as described in WO2006/056013 (PCT/AU2005/001781) the contents of which are incorporated by reference.

In a preferred embodiment, the phosphopeptide stabilized amorphous calcium phosphate (ACP) or amorphous calcium fluoride phosphate (ACFP) complex has tightly bound and loosely bound calcium, wherein the bound calcium in the complex is less than the tightly bound calcium in an ACP or ACFP complex formed at a pH of 7.0. Optionally, the ACP or ACFP is predominantly in a basic form.

A stabilized-ACP or ACFP complex as referred to herein include a stabilized-ACP or ACFP complex formed at a pH of below 7.0. Preferably, the complex is formed at a pH in the range of about 5.0 up to but below 7.0. More preferably, the complex is formed at a pH range of about 5.0 to about 6.0. In a preferred embodiment, the complex is formed at a pH of about 5.0 or about 5.5. Preferably, the ACP or ACFP in the complex is predominantly in a basic form.

A stabilized-ACP may be produced by a method comprising the steps of:

(i) obtaining a solution comprising at least one phosphopeptide and;

(ii) admixing solutions comprising calcium ions, phosphate ions and hydroxide ions, while maintaining the pH at about 5.5 to 9.

In one embodiment, the pH is maintained at 7.0 or below.

A stabilized ACFP may be produced by a method comprising the steps of:

(i) obtaining a solution comprising at least one phosphopeptide; and

(ii) admixing solutions comprising calcium ions, phosphate ions, hydroxide ions and fluoride ions, while maintaining the pH at about 5.5 to 9.

In one embodiment, the pH is maintained at 7.0 or below.

The hydroxide ions may be titrated into the solution to maintain the phosphopeptide solution at an essentially constant pH. The calcium and phosphate ions may be titrated into the phosphopeptide solution with constant mixing and at a rate that avoids the formation of a calcium phosphate precipitate in the phosphopeptide solution.

A phosphopeptide stabilized amorphous calcium phosphate (ACP) or amorphous calcium fluoride phosphate (ACFP) complex may also include wherein the ACP in the complex has tightly bound and loosely bound calcium, wherein the tightly bound calcium in the complex is less than the tightly bound calcium in an ACP or ACFP complex formed at a pH of 7.0 and the ACP or ACFP is predominantly in a basic form, obtainable or obtained by a method comprising:

a) admixing a first solution comprising calcium ions, a second solution comprising phosphate ions, and optionally a third solution comprising fluoride ions, to a solution comprising phosphopeptides and a solvent with a pH of from about 5 up to but below 7; and

b) maintaining the pH of the solution at about 5.0 up to but below 7.0 during the admixing by adding hydroxide ions.

“Tightly” and “loosely” bound calcium and phosphate can be determined using analytical ultrafiltration. Briefly, the solution of phosphopeptide, calcium, phosphate and optionally fluoride admixed while maintaining the pH at about 7.0 or below can be first filtered through a 0.1 micron filter to remove free calcium and phosphate that is not associated with the complexes. This free calcium and phosphate is present in the filtrate and discarded. Any free calcium or phosphate that is not associated in any way with the complexes would not be bioavailable, i.e. delivered by the phosphopeptide to the tooth. The retentate from the 0.1 micron filtration can be further analyzed by centrifugation through a 3000 mw cutoff filter at 1,000 g for 15 min. The resulting filtrate contains calcium and phosphate that is loosely bound or associated with the complexes. At this centrifugal force calcium and phosphate that is not tightly bound to the complexes are released and move into the filtrate. The Ca and Pi that is tightly bound in the complexes is retained in the retentate. The amount of tightly bound Ca and Pi in the retentate can then be determined by subtracting the amount of Ca and Pi in the filtrate from the total amount of Ca and Pi in the retentate of the 0.1 micron filtration.

A stabilized-ACP or ACFP complex as referred to herein include a stabilized-ACP or ACFP complex as described in WO2006/135982 (PCT/AU2006/000885) the contents of which are incorporated by reference.

A “superloaded” phosphopeptide or phosphoprotein (PP) stabilized-amorphous calcium phosphate (ACP) or amorphous calcium fluoride phosphate (ACFP) complex.

The complex may be formed at any pH (e.g. 3-10). Preferably the phosphopeptide includes the sequence -A-B-C-, where A is a phosphoamino acid, preferably phosphoserine, B is any amino acid including a phosphoamino acid and C is glutamic acid, aspartic acid or a phosphoamino acid. The phosphoamino acid may be phosphoserine. The PP is superloaded with calcium and phosphate ions. The calcium ions may be in the range 30-1000 mole Ca per mole of PP, or in the range of 30-100 or 30-50 mole Ca per mole of PP. In another embodiment, the mole Ca per mole of PP is at least 25, 30, 35, 40, 45 or 50.

The present invention includes a phosphopeptide or phosphoprotein (PP) stabilized amorphous calcium phosphate or amorphous calcium fluoride phosphate complex having a calcium ion content greater than about 30 moles of calcium per mole of PP. In a preferred embodiment, the calcium ion content is in the range of about 30 to 100 moles of calcium per mole of PP. More preferably, the calcium ion content is in the range of about 30 to about 50 moles of calcium per mole of PP.

The invention also provides a phosphopeptide or phosphoprotein (PP) stabilized-amorphous calcium phosphate (ACP) or amorphous calcium fluoride phosphate (ACFP) complex produced by a method comprising the steps of:

(i) obtaining solutions comprising calcium, inorganic phosphate and fluoride (optional); and
(ii) admixing (i) with a solution comprising PP-ACP.

In a preferred embodiment, the PP is casein phosphopeptide (CPP).

In a further aspect, the present invention also includes use of a formulation of a PP stabilized ACP and/or ACFP complex together with at least an equal amount by weight of calcium phosphate. Preferably, the calcium phosphate is CaHPO4 or calcium lactate or any other soluble calcium phosphate compound. Preferably, the calcium phosphate (e.g. CaHPO₄) is dry blended with the PP stabilized ACP and/or ACFP complex. In a preferred embodiment, the PP-ACP and/or PP-ACFP complex: calcium phosphate ratio is about 1:1-50, more preferably about 1:1-25, more preferably about 1:5-15. In one embodiment, the PP-ACP and/or PP-ACFP complex: calcium phosphate ratio is about 1:10.

The oral care formulation that includes a phosphopeptide or phosphoprotein (PP) stabilized amorphous calcium phosphate (ACP) and/or amorphous calcium fluoride phosphate (ACFP) complex having a calcium ion content greater than about 30 moles of calcium per mole of PP when used in the oral cavity may be produced by a method including the steps of:

(i) obtaining a powder including a PP-ACP and/or PP-ACFP complex;
(ii) dry blending with an effective amount of calcium phosphate; and
(iii) formulating the dry blended PP-ACP and/or PP-ACFP and calcium phosphate mixture into an oral care formulation.

Preferably, the form of calcium phosphate for dry blending is any soluble calcium phosphate including, but not limited to, CaHPO₄, Ca₂HPO₄ and calcium lactate.

A composition as described herein may further include free fluoride ions. The fluoride ions may be from any suitable source. A source of fluoride ions may include free fluoride ions or fluoride salts. Examples of sources of fluoride ions include, but are not limited to the following: sodium fluoride, sodium monofluorophosphate, stannous fluoride, sodium silicofluoride and amine fluoride. These may be provided in solution (typically an aqueous solution), or a suspension.

The fluoride ions are preferably present in the composition in an amount greater than 1 ppm. More preferably, the amount is more than 3 ppm. In another embodiment, it is preferably more than 10 ppm. In typical embodiments described below, the amount may be several hundred or thousand ppm. The fluoride content is typically measured as a ppm in oral compositions in the manner commonly used in the art. Where the fluoride is provided from a source with the stabilized ACP, the ppm refers to the concentration of the fluoride in that source, typically a solution or suspension of bioavailable fluoride.

A stannous-associated ACP or ACFP complex as referred to herein include any described in PCT/AU2014/050447, the entire contents of which are incorporated by reference in its entirety.

A composition as described herein for use in a method of use of the invention may include a stannous-associated ACP or ACFP complex. The composition may include 2% CPP-ACP and 290 ppm fluoride with 220 ppm fluoride as stannous fluoride and 70 ppm as sodium fluoride.

In any aspect or embodiments as described herein, the stabilized ACP and/or ACFP is phosphopeptide (PP)-stabilized. Preferably, the phosphopeptide (as defined below) is a casein phosphopeptide. Preferably, the ACP or ACFP is in the form of a casein phosphopeptide stabilized ACP or ACFP complex.

“Phosphopeptide” in the context of the description of this invention means an amino acid sequence in which at least one amino acid is phosphorylated. Preferably, the phosphopeptide includes one or more of the amino acid sequence -A-B-C-, where A is a phosphoamino residue, B is any amino acyl residue including a phosphoamino residue and C is selected from a glutamyl, aspartyl or phosphoamino residue. Any of the phosphoamino residues may independently be a phosphoseryl residue. B is desirably a residue the side-chain of which is neither relatively large nor hydrophobic. It may be Gly, Ala, Val, Met, Leu, Ile, Ser, Thr, Cys, Asp, Glu, Asn, Gln or Lys. Preferably, at least two of the phosphoamino acids in the sequence are preferably contiguous. Preferably, the phosphopeptide includes the sequence A-B-C-D-E, where A, B, C, D and E are independently phosphoserine, phosphothreonine, phosphotyrosine, phosphohistidine, glutamic acid or aspartic acid, and at least two, preferably three, of the A, B, C, D and E are a phosphoamino acid. In a preferred embodiment, the phosphoamino acid residues are phosphoserine, most preferably three contiguous phosphoserine residues. It is also preferred that D and E are independently glutamic or aspartic acid.

In one embodiment, the ACP or ACFP is stabilized by a casein phosphopeptide (CPP), which is in the form of intact casein or fragment of the casein, and the complex formed preferably has the formula [CPP(ACP)₈]ₙ or [(CPP)(ACFP)₈]_{n w}here n is equal to or greater than 1, for example 6. The complex formed may be a colloidal complex, where the core particles aggregate to form large (e.g. 100 nm) colloidal particles suspended in water. Thus, the PP can be a casein protein or a phosphopeptide.

The PP may be from any source; it may be present in the context of a larger polypeptide, including a full length casein polypeptide, or it may be isolated by tryptic or other enzymatic or chemical digestion of casein, or other phosphoamino acid rich proteins such as phosphitin, or by chemical or recombinant synthesis, provided that it comprises the sequence -A-B-C- or A-B-C-D-E as described above. The sequence flanking this core sequence may be any sequence. However, those flanking sequences in αₛ₁(59-79), β(1-25), αₛ₂(46-70) and αₛ₂(1-21) are preferred. The flanking sequences may optionally be modified by deletion, addition or conservative substitution of one or more residues. The amino acid composition and sequence of the flanking region are not critical.

The phosphopeptide may be selected from any described in WO2006/056013, WO2006/135982 or U.S. Pat. No. 5,015,628.

Examples of conservative substitutions are shown in Table 1 below.

TABLE 1

| Original | Exemplary Conservative | Preferred Conservative |
|---|---|---|
| Residue | Substitution | Substitution |
| Ala | Val, Leu, Ile | Val |
| Asn | Gln Lys His Phe | Gln |
| Gln | Asn | Asn |
| Gly | Pro | Pro |
| Ile | Leu, Val, Met, Ala, Phe | Leu |
| Leu | Ile, Val, Met, Ala, Phe | Ile |
| Lys | Arg, Gln, Asn | Arg |
| Phe | Leu, Val, Ile, Ala | Leu |
| Pro | Gly | Gly |
| Ser | Thr | Thr |
| Val | Ile, Leu, Met, Phe, Ala | Leu |
| Asp | Glu | Glu |
| Thr | Ser | Ser |
| Trp | Tyr | Tyr |
| Tyr | Trp Phe Thr Ser | Phe |

The flanking sequences may also include non-naturally occurring amino acid residues. Commonly encountered amino acids which are not encoded by the genetic code, include:

2-amino adipic acid (Aad) for Glu and Asp;

2-aminopimelic acid (Apm) for Glu and Asp;

2-aminobutyric (Abu) acid for Met, Leu, and other aliphatic amino acids;

2-aminoheptanoic acid (Ahe) for Met, Leu and other aliphatic amino acids;

2-aminoisobutyric acid (Aib) for Gly;

cyclohexylalanine (Cha) for Val, and Leu and Ile;

homoarginine (Har) for Arg and Lys;

2, 3-diaminopropionic acid (Dpr) for Lys, Arg and His;

N-ethylglycine (EtGly) for Gly, Pro, and Ala;

N-ethylasparigine (EtAsn) for Asn, and Gln;

Hydroxyllysine (Hyl) for Lys;

allohydroxyllysine (AHyl) for Lys;

3-(and 4) hydroxyproline (3Hyp, 4Hyp) for Pro, Ser, and Thr;

alloisoleucine (Alle) for Ile, Leu, and Val;

ρ-amidinophenylalanine for Ala;

N-methylglycine (MeGly, sarcosine) for Gly, Pro, Ala.

N-methylisoleucine (Melle) for Ile;

Norvaline (Nva) for Met and other aliphatic amino acids;

Norleucine (Nle) for Met and other aliphatic amino acids;

Ornithine (Orn) for Lys, Arg and His;

Citrulline (Cit) and methionine sulfoxide (MSO) for Thr, Asn and Gln;

N-methylphenylalanine (MePhe), trimethylphenylalanine, halo (F, CI, Br and I) phenylalanine, triflourylphenylalanine, for Phe.

In one embodiment, the PP is one or more phosphopeptides selected from the group consisting of αₛ₁(59-79) [1], β(1-25) [2], αₛ₂(46-70) [3] and αₛ₂(1-21) [4]:

(SEQ ID NO: 1)
[1] Gln⁵⁹-Met-Glu-Ala-Glu-Ser(P)-Ile-Ser(P)-
Ser(P)-Ser(P)-Glu-Glu-Ile-Val-
Pro-Asn-Ser(P)-Val-Glu-Gln-Lys⁷⁹ αₛ₁(59-79)
(SEQ ID NO: 2)
[2] Arg^{l}-Glu-Leu-Glu-Glu-Leu-Asn-Val-
Pro-Gly-Glu-Ile-Val-Glu-Ser(P)-Leu-
Ser(P)-Ser(P)-Ser(P)-Glu-Glu-Ser-
Ile-Thr-Arg²⁵ β(1-25)
(SEQ ID NO: 3)
[3] Asn⁴⁶-Ala-Asn-Glu-Glu-Glu-Tyr-Ser-Ile-
Gly-Ser(P)-Ser(P)-Ser(P)-Glu-Glu-
Ser(P)-Ala-Glu-Val-Ala-Thr-Glu-Glu-
Val-Lys⁷⁰ αₛ₂(46-70)
(SEQ ID NO: 4)
[4] Lys¹-Asn-Thr-Met-Glu-His-Val-Ser(P)-
Ser(P)-Ser(P)-Glu-Glu-Ser-Ile-Ile-
Ser(P)-Gln-Glu-Thr-Tyr-Lys²¹ αₛ₂(1-21).

In certain preferred forms of the invention a liquid composition may be a mouthwash, rinse or spray. In such a preparation the vehicle is typically a water-alcohol mixture desirably including a humectant. Generally, the weight ratio of water to alcohol is in the range of from about 1:1 to about 20:1. The total amount of water-alcohol mixture in this type of preparation is typically in the range of from about 70 to about 99.9% by weight of the preparation. The alcohol is typically ethanol or isopropanol. Ethanol is preferred.

It will be understood that, as is conventional, the oral preparations will usually be sold or otherwise distributed in suitable labelled packages. Thus, a jar of mouth rinse will have a label describing it, in substance, as a mouth rinse or mouthwash and having directions for its use.

Prior to addition of the liquid composition the dental surface, subsurface or lesion may be prepared (e.g. cleaned) using preparative compositions. Such compositions may include the following components. Organic surface-active agents may be used in the compositions to achieve increased prophylactic action, assist in achieving thorough and complete dispersion of the active agent throughout the oral cavity, and render the instant compositions more cosmetically acceptable. The organic surface-active material is preferably anionic, non-ionic or ampholytic in nature and preferably does not interact with the active agent. It is preferred to employ as the surface-active agent a detersive material which imparts to the composition detersive and foaming properties. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkylsulfo-acetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarconite compounds in the oral compositions of the present invention is particularly advantageous since these materials exhibit a prolonged marked effect in the inhibition of acid formation in the oral cavity due to carbohydrate breakdown in addition to exerting some reduction in the solubility of tooth enamel in acid solutions. Examples of water-soluble non-ionic surfactants suitable for use are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly (ethylene oxide) with fatty acids, fatty alcohols, fatty amides, polyhydric alcohols (e.g. sorbitan monostearate) and polypropyleneoxide (e.g. Pluronic materials).

Various other materials may be incorporated in the oral preparations of this invention such as whitening agents, preservatives, silicones, chlorophyll compounds and/or ammoniated material such as urea, diammonium phosphate, and mixtures thereof. These adjuvants, where present, are incorporated in the preparations in amounts which do not substantially adversely affect the properties and characteristics desired. Any suitable flavouring or sweetening material may also be employed. Examples of suitable flavouring constituents are flavouring oils, e.g. oil of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, xylitol, sodium cyclamate, perillartine, AMP (aspartyl phenyl alanine, methyl ester), saccharine, and the like. Suitably, flavour and sweetening agents may each or together comprise from about 0.1% to 5% more of the preparation.

The further composition of alkaline pH may further comprise additional components to enhance gel formation. For example the addition of stannous, zinc, magnesium or other metal ions or other chemicals which help cross-link the phosphopeptide-stabilized ACP or ACFP to enhance gelation.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals.

The invention will now be further described with reference to the following non-limiting examples.

### EXAMPLE 1

### Preparation of High Concentration Liquid CPP-ACFP and CPP-ACP Solutions

Stock solutions of 3.25 M CaCl₂ and 1.25 M NaH₂PO₄ (pH 5.5) were added in approximately thirty aliquots to a 10-15% w/v tryptic digest of casein until just before precipitation or gelation (usually producing a final concentration of approximately 78 mM to 124 Ca²⁺ and 48 to 76 mM inorganic phosphate). The solutions were added slowly (that is, less than approximately 1% volume addition per minute) with adequate mixing. An aliquot of the phosphate solution was added first, followed by an aliquot of the calcium solution. The bulk solution pH was maintained at 9.0 using 1 to 10 M NaOH with thorough mixing. The sodium hydroxide solution was added automatically by a pH stat with the addition of the hydroxide ions usually being after each addition of the calcium ions. After completion of the addition of the calcium ions, phosphate ions and hydroxide ions the solution was filtered through a 0.1 micron filter to concentrate 1-2 fold. The retentate was then washed with 1-2 volumes of water to remove salts and inactive (and bitter tasting) peptides. The CPP-ACP solutions prepared were then spray dried or freeze dried to produce a white powder. This dried powder was then added to water to form a 45% w/v CPP-ACP solution at pH 5.5 by addition of 1-10 M HCl or a 63% w/v CPP-ACP solution with added NaF to produce 8,200 ppm F at pH 5.5 by addition of 1-10 M HCl.

The 75% w/v solution was prepared by adding 75 g CPP-ACP powder to 20 ml water with a small amount of powder each addition (0.5 g/min) while maintaining the pH at 5.5 by the addition of 10 M HCl. The solution was thoroughly mixed after each addition to ensure dispersion. A concentrated NaF (0.95 M) solution was added together with 10 M HCl to ensure that 52 mmol of F was finally added. The CPP-ACP powder, NaF and HCl were added over 2-3 hours with water to a final volume of 100 ml. This produced a liquid composition of 75% w/v CPP-ACP, 9,880 ppm F at pH 5.5.

### EXAMPLE 2

### Masking a White Spot Lesion Using CPP-ACP and Alkaline Solution

A 45% w/v CPP-ACP pH 5.5 liquid solution was applied with a microbrush (for a few seconds) to the surface of an enamel block with a white spot lesion. The enamel block was then incubated for 20 minutes at 37° C. Then a 1 M NaOH solution (about pH 14) was applied with a microbrush (for a few seconds) and the enamel block was then incubated at 37° C. for a further 20 minutes.

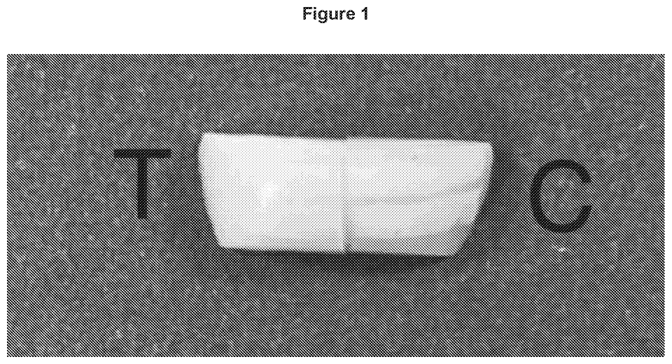

FIG. 1 shows the white spot lesions after treatment (T) compared with control lesions (C), which were derived from the same lesions (i.e. enamel block cut into two).

The white spot lesions in the treatment sample were substantially masked by the treatment by returning the lesion translucent compared to the control sample where the white spot lesions are still clearly visible.

### EXAMPLE 3

### Masking a White Spot Lesion Using 63% w/v CPP-ACP, Free Fluoride and Alkaline Solution

A 63% w/v CPP-ACP and 8,200 ppm F as NaF pH 5.5 liquid solution was applied with a microbrush (for a few seconds) to the surface of an enamel block with a white spot lesion. The enamel block was then incubated for 20 minutes at 37° C. Then a 1 M NaOH solution was applied with a microbrush (for a few seconds) and the enamel block was then incubated at 37° C. for a further 20 minutes.

FIG. 2 shows the white spot lesions after treatment (T) on the left compared with control lesions (C) on the right, which were derived from the same lesions (i.e. enamel block cut into two). The white spot lesions in the treatment sample were substantially masked by the treatment by returning the lesion translucent compared to the control sample where the white spot lesions are still clearly visible.

### EXAMPLE 4

In a dental clinic, a patient in need of a reduction in the visibility of a hypomineralised dental surface or subsurface of the tooth enamel may be treated using the steps of:

1. Apply rub dam and acid etch the white spot [this step is optional and not necessary for very porous (active) lesions]. This can involve standard liquid rubber dams (resin barrier to protect soft tissue) and acid etching techniques (e.g. 30% phosphoric acid or 15% HCl).
2. Apply high concentration (>40% w/v) stabilised CPP-ACP or CPP-ACP/F acid solutions (e.g. less than pH 6) to white spots using a microbrush and then leave for 5-20 min. Heat can be optionally applied by using a high intensity LED curing light (e.g. 1 Ow high power blue light) to increase temperature in bursts to 45-50° C. (with patient comfort).
3. Apply high concentration base (e.g. 4% w/v NaOH) using a microbrush and then leave for 5-20 min. Heat can be optionally applied by using a high intensity LED curing light (e.g. 10W high power blue light) to increase temperature in bursts to 45-50° C. (with patient comfort).

### EXAMPLE 5

### Exemplary Dental Kit

A dental kit comprising or consisting of three parts:

(a) 5 g of a CPP-ACP powder, preferably prepared as described herein,

(b) 5 ml of 0.73 M NaF in 1.146 M HCl, and

(c) 1.5 M NaOH, including two microbrushes.

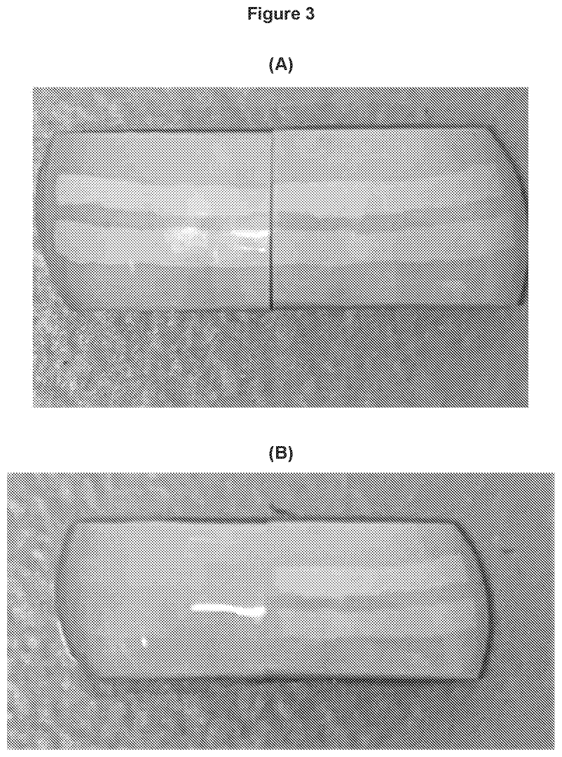

Part (a) was added to part (b) with thorough mixing. This mixture was then applied with one microbrush to the white spot lesions on the left (see image in FIG. 3 (A) showing lesions before application). After a few secs/mins at 37° C. solution (c) was then be applied to the white spot lesions on the left with the second microbrush. Within 15 mins the reaction in the white spot occurred to form a gel and concealed the white spots (see image in FIG. 3(B)).

### EXAMPLE 6

### Masking a White Spot Lesion Using 75% w/v CPP-ACP, Free Fluoride and Alkaline Solution

A 75% w/v CPP-ACP and 9,880 ppm F as NaF pH 5.5 liquid was applied with a microbrush (for a few seconds) to the surface of an enamel block with a white spot lesion. The enamel block was then incubated for 10 minutes at 37° C. Then a 2M NaOH solution was applied with a microbrush (for a few seconds) and the enamel block was then incubated at 37° C. for a further 20 minutes.

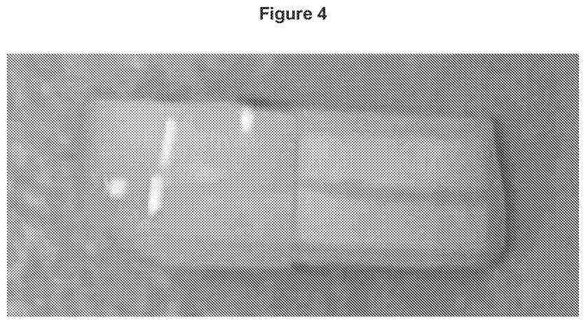

FIG. 4 shows the white spot lesions after treatment on the left compared with control lesions on the right, which were derived from the same lesions (i.e. enamel block cut into two). The white spot lesions in the treatment sample were substantially masked by the treatment by returning the lesion translucent compared to the control sample where the white spot lesions are still clearly visible.

### EXAMPLE 7

### Masking a White Spot Lesion by Forming Mixed Composition Prior to Application to the Dental Surface

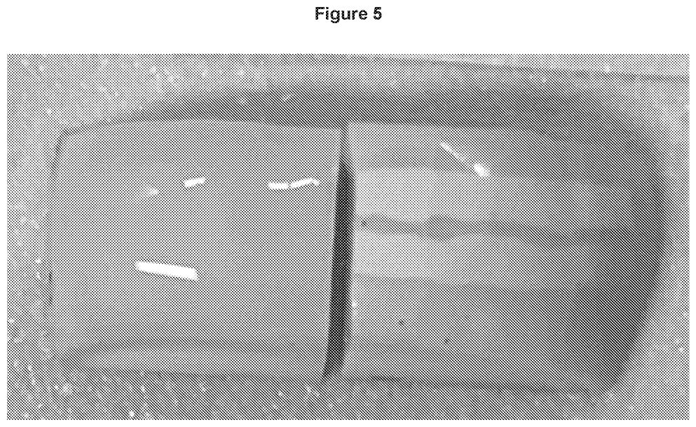

A liquid composition of a degassed 63% w/v CPP-ACP/ 8,000 ppm F at pH 5.5 was mixed with a solution of 1.5 M NaOH prior to application to a dental surface. The mixed composition was then painted on to a white spot lesion. A dental curing light on setting 2 was then applied to the surface for 40 secs. The result is an impressive covering of the enamel white spots (FIG. 5).

### EXAMPLE 8

### Formation of a Protective Layer Over Dentine

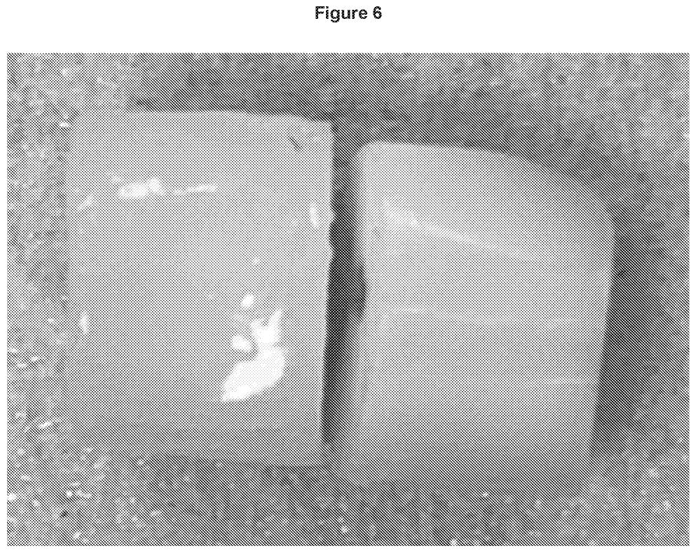

Another application of this invention is to seal or occlude exposed tooth root (dentine) surfaces (aging population has exposed root surfaces and they are more susceptible to caries/erosion). To simulate these exposed root surfaces tooth root dentine was treated with 15% EDTA for 2 min thereby removing the smear layer to expose dentinal tubules (FIG. 6, right block). Applying the mixed composition as described in Example 7 to the dentine resulted in formation of a gel on the surface (FIG. 6, left block).

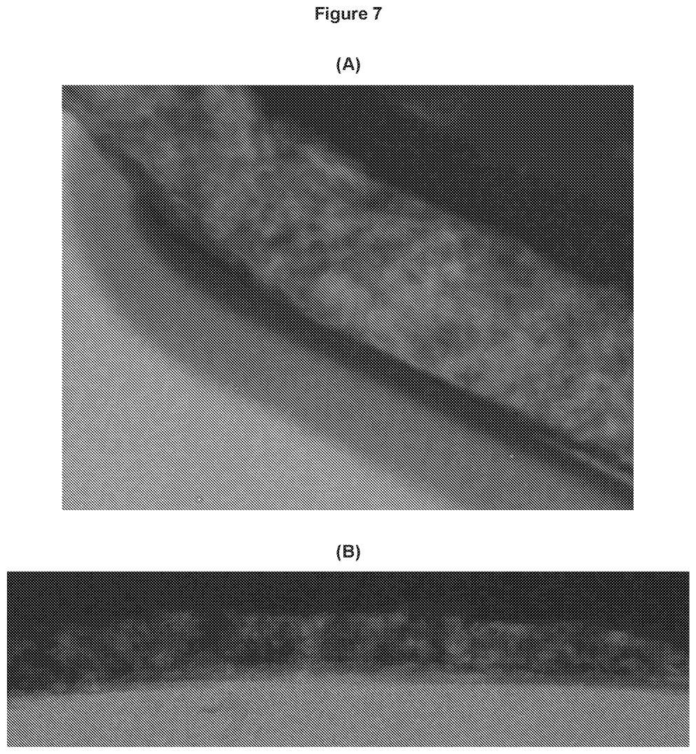

Further, when the mixed composition was applied to the dentine surface (either sound or prior demineralised with acid buffer) the solution not only gels but also starts to form a fluorapatite (FA) layer on the surface to seal the dentine (shown in FIGS. 7, 8 and 9). This would have a dramatic effect at reducing tooth sensitivity and also reducing the risk of root caries. Interestingly, the fluorapatite layer is white so conceals the yellow dentine thereby not only providing a seal and protective layer to reduce sensitivity and caries/erosion risk but also improving the aesthetic of the tooth.

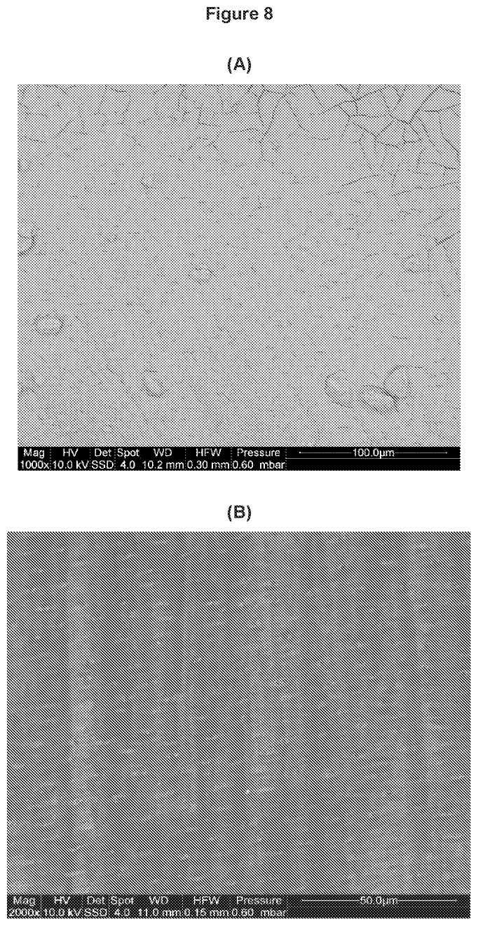

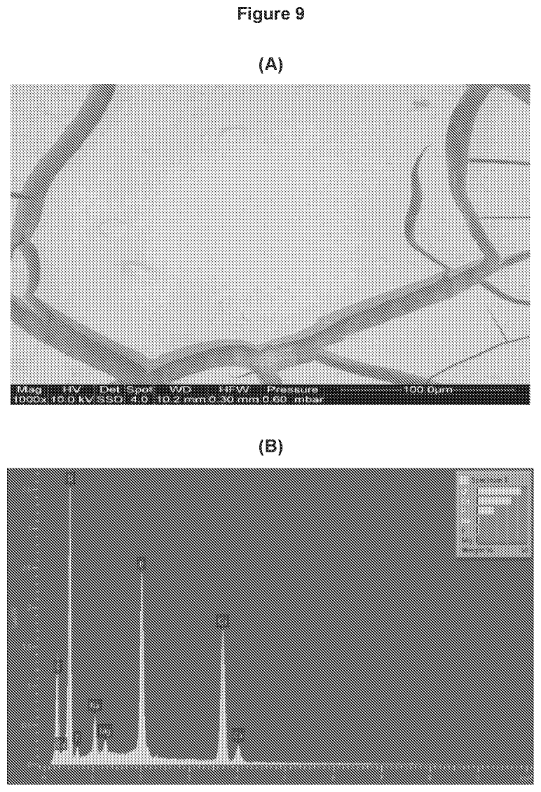

The protective layer formed was purposefully dehydrated (as shown in FIG. 9) to demonstrate its uniformity on the surface which had completely sealed the dentine tubules (shown exposed on the right in FIG. 8).

An elemental analysis revealed that the formed protective layer had a composition similar to fluorohydroxyapaptite (FIG. 9B).

### EXAMPLE 9

### Formation of a protective layer over dentine

### Metho:

All dentine blocks were polished

Dentine block treated with 15% EDTA for 2 min

15 second etch (37% phosphoric acid)

Mix degassed 63% (w/v) CPP-ACP/8,100 ppm F (pH 5.5) with equal volume of 2 M NaOH

Topical application of pre-mixed 63% (w/v) CPP-ACP/8,100 ppm F (pH 5.5) and 2 M NaOH on dentine surface using microbrush

40 second light cure

Leave at 37° C. for 48 h

Sectioned, lapped and microradiographed.

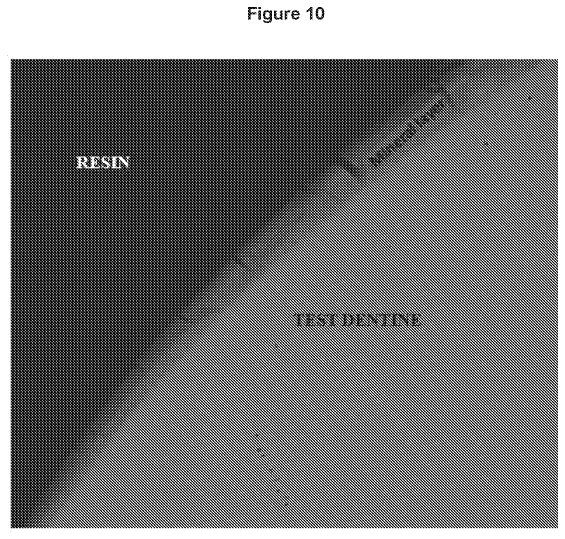

As shown in FIG. 10, a mineral layer on the dentine surface was formed.

### EXAMPLE 10

### Formation of a Protective Layer Over Enamel

### Method

All enamel blocks were polished

15 second etch (37% phosphoric acid)

Mix 63% (w/v) CPP-ACP/8100ppm F (pH 5.5) with equal volume of 2 M NaOH

Topical application of pre-mixed 63% (w/v) CPP-ACP/8,100 ppm F (pH 5.5) and 2 M NaOH on enamel surface using microbrush

40 second light cure

Leave at 37° C. for 48 h

Sectioned, lapped and microradiographed.

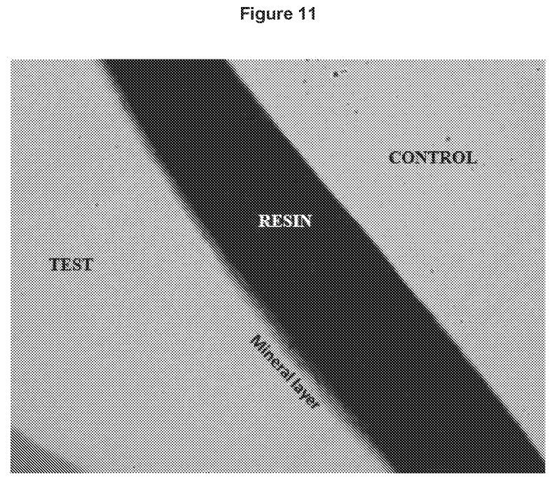

As shown in FIG. 11, a mineral layer on the enamel surface was formed.

### EXAMPLE 11

### Masking a White Spot Lesion by Forming Mixed Composition Prior to Application to the Dental Surface

30 g of CPP-ACP powder (commercial Recaldent) was added to 19.5 g of a 20,000 ppm F (NaF) solution to which 0.5 g of an 11 M HCl solution was added to give the final weight 50 g (hence this final solution is a 60% w/w CPP-ACP with 7,800 ppm F at pH 7.8 or 75% w/v CPP-ACP containing 10,000 mg/L F at pH 7.8).

With thorough stirring (around 30 min) a homogeneous very viscous but stable solution was prepared with a pH of 7.8. This solution was then degassed to remove trapped air bubbles by placing the solution under vacuum for 24 hours.

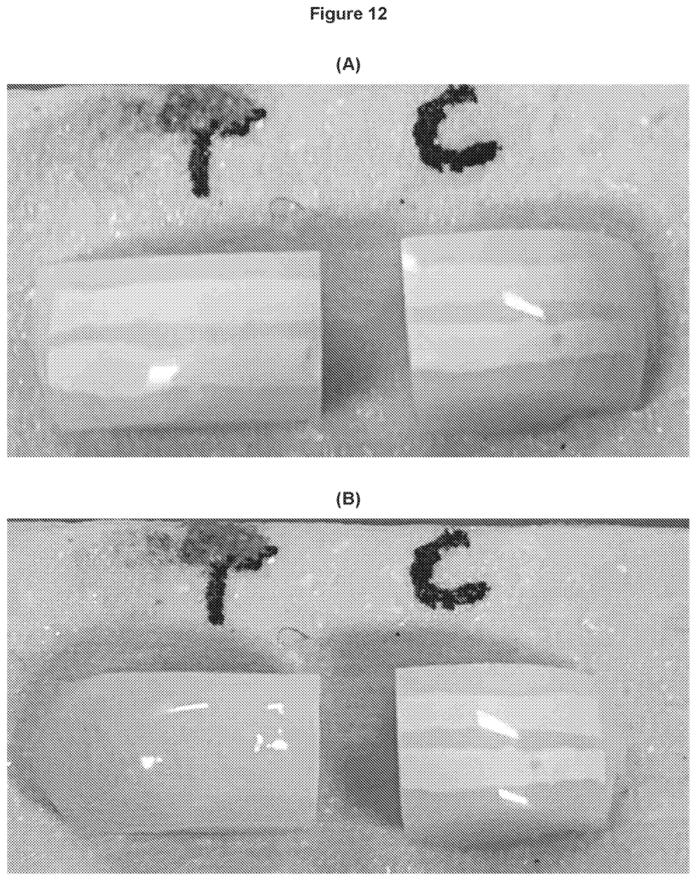

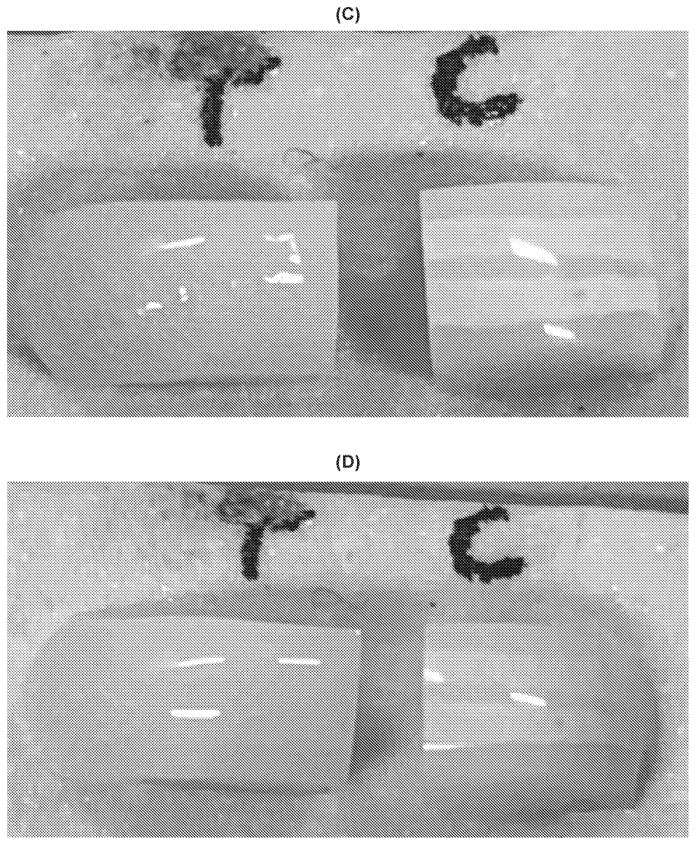

The solution was applied to white spot lesions using a microbrush and then light cured for 40 sec using the dental curing light (as described previously). The images of before and after (FIG. 12(a) before, FIG. 12(b) after 40 sec of curing, FIG. 12(c) 15 min after curing and then FIG. 12(d) 24 hours after curing) are shown. The cured stabled high concentration solution conceals the white spots very effectively and the effect was maintained for at least 24 hours at 37° C.

The viscous, stable and safe (neutral pH) solution is easy to apply in the dental surgery and is more concentrated so produces a better effect over a longer period of time.

It will be understood that the invention disclosed and defined in this specification extends to all alternative combinations of two or more of the individual features mentioned or evident from the text or drawings. All of these different combinations constitute various alternative aspects of the invention.

1. A method of reducing visibility of a hypomineralised dental surface or subsurface, the method comprising
contacting the hypomineralised dental surface or subsurface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,
thereby reducing visibility of a hypomineralised dental surface or subsurface.

2. A method of treating or preventing dentinal sensitivity in a subject in need thereof, the method comprising:
contacting exposed dentinal tubules with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 5 but less than or equal to pH 9,
thereby treating or preventing dentinal sensitivity in the subject in need thereof.

3. A method according to claim 1 or 2, wherein the pH of the liquid composition is greater than or equal to 6 but less than or equal to 8, preferably, greater than or equal to 7 but less than or equal to 8.

4. A method according to any one of claims 1 to 3, wherein the liquid composition further comprises free fluoride ions.

5. A method according to claim 4, wherein the free fluoride ions are present in the liquid composition at a concentration in the range of about 200 ppm to 10,000 ppm.

6. A method according to claim 4, wherein the free fluoride ions are present in the liquid composition at a concentration in the range of about 2,600 ppm to about 8,500 ppm.

7. A method according to claim 4, wherein the free fluoride ions are present in the liquid composition at a concentration of about 7,800 ppm.

8. A method according to any one of claims 1 to 7, the method further comprising heating of the dental surface or subsurface, or lesion.

9. A method according to claim 8, wherein the dental surface or subsurface is heated to a temperature greater than 37° C.

10. A method according to claim 8, wherein the dental surface or subsurface is heated to a temperature greater than or equal to 40° C.

11. A method according to claim 8, wherein the dental surface or subsurface is heated to a temperature greater than or equal to 45° C.

12. A method according to claim 8, wherein the dental surface or subsurface is heated to a temperature greater than or equal to 50° C.

13. A method according to claim 8, wherein the dental surface or subsurface is heated to a temperature greater than or equal to 55° C.

14. A method according to claim 9, wherein the dental surface or subsurface is heated to a temperature greater than or equal to 60° C.

15. A method according to claim 9, wherein the dental surface or subsurface is heated to a temperature greater than or equal to 65° C.

16. A method according to any one of claims 1 to 15, wherein the liquid composition comprises greater than 45% w/w stabilized ACP and/or ACFP.

17. A method according to any one of claims 1 to 15, wherein the liquid composition comprises greater than 50% w/w stabilized ACP and/or ACFP.

18. A method according to any one of claims 1 to 15, wherein the liquid composition comprises greater than 55% w/w stabilized ACP and/or ACFP.

19. A method according to any one of claims 1 to 15, wherein the liquid composition comprises greater than 60% w/w stabilized ACP and/or ACFP.

20. A method according to any one of claims 1 to 15, wherein the liquid composition comprises about 65% w/v stabilized ACP and/or ACFP.

21. A method according to any one of claims 1 to 15, wherein the liquid composition comprises about 70% w/v stabilized ACP and/or ACFP.

22. A method according to any one of claims 1 to 15, wherein the liquid composition comprises about 75% w/v stabilized ACP and/or ACFP.

23. A method according to any one of claims 1 to 22, wherein the phosphopeptide is a casein phosphopeptide.

24. A method of reducing visibility of a hypomineralised dental surface or subsurface, the method comprising:
(i) contacting the hypomineralised dental surface or subsurface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and
(ii) subsequently to (i), raising the pH of the liquid composition applied to the hypomineralised dental surface or subsurface to equal to, or greater than, about 9, thereby forming a gel in and/or on the hypomineralised dental surface or subsurface,
thereby reducing visibility of a hypomineralised dental surface or subsurface.

25. A method of forming a gel in and/or on a dental surface or sub-surface lesion, the method comprising:
(i) contacting the dental surface or sub-surface lesion with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and
(ii) subsequently to (i), raising the pH of the liquid composition applied to the dental surface or sub-surface lesion to equal to, or greater than, about 9, thereby forming a gel in and/or on the dental surface or sub-surface lesion,
thereby forming a gel in and/or on the dental surface or sub-surface lesion.

26. A method of treating or preventing dentinal sensitivity in a subject in need thereof, the method comprising:
(i) contacting exposed dentinal tubules with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and
(ii) subsequently to (i), raising the pH of the liquid composition applied to the hypomineralised dental surface or subsurface to equal to, or greater than, about 9, thereby forming a gel in and/or on the exposed dentinal tubules,
thereby treating or preventing dentinal sensitivity in the subject in need thereof.

27. A method of forming a protective layer on a dental surface, the method comprising:
(i) contacting the dental surface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and
(ii) subsequently to (i), raising the pH of the liquid composition applied to the dental surface to equal to, or greater than, about 9,
thereby forming a protective layer on the dental surface.

28. A method according to any one of claims 24 to 27, wherein the pH of the liquid composition applied to the dental surface or sub-surface lesion is raised to equal to, or greater than, about 10.

29. A method according to any one of claims 24 to 27, wherein raising the pH of the liquid composition applied to the dental surface or sub-surface is by contacting the liquid composition applied to the dental surface or sub-surface with a further composition of alkaline pH.

30. A method according to any one of claims 24 to 29, wherein the dental surface is dental enamel.

31. A method according to any one of claims 24 to 30, wherein the dental surface is a lesion in enamel is caused by caries, dental erosion or fluorosis.

32. A method according to claim 31, wherein the lesion is a white spot lesion.

33. A method according to any one of claims 24 to 32, wherein raising the pH of the liquid composition applied to the dental surface or sub-surface is by contacting the liquid composition applied to the dental surface or sub-surface with a further composition of alkaline pH.

34. A method of reducing visibility of a hypomineralised dental surface or subsurface, the method comprising:
(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;
(ii) contacting the hypomineralised dental surface or subsurface with the mixed composition, thereby forming a gel in and/or on the hypomineralised dental surface or subsurface,
thereby reducing visibility of a hypomineralised dental surface or subsurface.

35. A method of treating or preventing dentinal sensitivity in a subject in need thereof, the method comprising:
(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;
(ii) contacting exposed dentinal tubules with the mixed composition, thereby forming a gel in and/or on the exposed dentinal tubules,
thereby treating or preventing dentinal sensitivity in the subject in need thereof.

36. A method of forming a protective layer on a dental surface, the method comprising:
(i) mixing (a) a liquid composition comprising at least 40% w/v of phosphopeptide (PP)-stabilized amorphous calcium phosphate (ACP) and/or amorphous calcium fluoride phosphate (ACFP) at a pH of less than or equal to pH 6, and (b) a further composition of alkaline pH, thereby forming a mixed composition with a pH of equal to, or greater than, about 9;
(ii) contacting a dental surface with the mixed composition,
thereby forming a protective layer on the dental surface.

37. A method according to claims 34 to 36, wherein the alkaline pH of the further composition is a pH of about 8.

38. A method according to claims 34 to 36, wherein the alkaline pH of the further composition is a pH of about 9.

39. A method according to claims 34 to 36, wherein the alkaline pH of the further composition is a pH of about 10.

40. A method according to claims 34 to 36, wherein the alkaline pH of the further composition is a pH of about 11.

41. A method according to claims 34 to 36, wherein the alkaline pH of the further composition is a pH of about 12.

42. A method according to claims 34 to 36, wherein the alkaline pH of the further composition is a pH of about 13.

43. A method according to claims 34 to 36, wherein the alkaline pH of the further composition is a pH of about 14.

44. A method according to any one of claims 24 to 43, wherein the liquid composition further comprises free fluoride ions.

45. A method according to claim 44, wherein the free fluoride ions are present in the liquid composition at a concentration in the range of about 200 ppm to about 10,000 ppm.

46. A method according to claim 44, wherein the free fluoride ions are present in the liquid composition at a concentration in the range of about 2,600 ppm to about 8,500 ppm.

47. A method according to claim 44, wherein the free fluoride ions are present in the liquid composition at a concentration of about 8,200 ppm.

48. A method according to any one of claims 24 to 33, wherein step (i) further comprises heating of the dental surface or subsurface, or lesion.

49. A method according to any one of claims 24 to 33, wherein step (i) is followed by heating of the dental surface or subsurface, or lesion.

50. A method according to any one of claims 24 to 49, wherein step (ii) further comprises heating of the dental surface or subsurface, or lesion.

51. A method according to any one of claims 24 to 49, wherein step (ii) is followed by heating of the dental surface or subsurface, or lesion.

52. A method according to any one of claims 48 to 51, wherein the dental surface or subsurface, or lesion is heated to a temperature greater than 37° C.

53. A method according to any one of claims 48 to 51, wherein the dental surface or subsurface, or lesion is heated to a temperature greater than or equal to 40° C.

54. A method according to any one of claims 48 to 51, wherein the dental surface or subsurface, or lesion is heated to a temperature greater than or equal to 45° C.

55. A method according to any one of claims 48 to 51, wherein the dental surface or subsurface, or lesion is heated to a temperature greater than or equal to 50° C.

56. A method according to any one of claims 48 to 51, wherein the dental surface or subsurface, or lesion is heated to a temperature greater than or equal to 55° C.

57. A method according to any one of claims 48 to 51, wherein the dental surface or subsurface, or lesion is heated to a temperature greater than or equal to 60° C.

58. A method according to any one of claims 48 to 51, wherein the dental surface or subsurface, or lesion is heated to a temperature greater than or equal to 65° C.

59. A method according to any one of claims 48 to 51, wherein the dental surface or subsurface, or lesion is heated to a temperature less than 65° C.

60. A method according to any one of claims 24 to 59, wherein the liquid composition comprising greater than 40% w/v phosphopeptide (PP)-stabilized ACP and/or ACFP comprises greater than 45% w/v stabilized ACP and/or ACFP.

61. A method according to any one of claims 24 to 59, wherein the liquid composition comprising greater than 40% w/v phosphopeptide (PP)-stabilized ACP and/or ACFP comprises greater than 50% w/v stabilized ACP and/or ACFP.

62. A method according to any one of claims 24 to 59, wherein the liquid composition comprising greater than 40% w/v phosphopeptide (PP)-stabilized ACP and/or ACFP comprises greater than 55% w/v stabilized ACP and/or ACFP.

63. A method according to any one of claims 24 to 59, wherein the liquid composition comprising greater than 40% w/v phosphopeptide (PP)-stabilized ACP and/or ACFP comprises greater than 60% w/v stabilized ACP and/or ACFP.

64. A method according to any one of claims 24 to 59, wherein the liquid composition comprising greater than 40% w/v phosphopeptide (PP)-stabilized ACP and/or ACFP comprises about 65% w/v stabilized ACP and/or ACFP.

65. A method according to any one of claims 24 to 59, wherein the liquid composition comprising greater than 40% w/v phosphopeptide (PP)-stabilized ACP and/or ACFP comprises about 70% w/v stabilized ACP and/or ACFP.

66. A method according to any one of claims 24 to 59, wherein the liquid composition comprising greater than 40% w/v phosphopeptide (PP)-stabilized ACP and/or ACFP comprises about 75% w/v stabilized ACP and/or ACFP.

67. A method according to any one of claims 24 to 33, wherein the hypomineralised dental surface or subsurface, or lesion, is contacted with the liquid composition in (i) for up to 20 minutes before the raising the pH of the liquid composition applied to the hypomineralised dental surface or subsurface to equal to, or greater than, about 9.

68. A method according to claim 67, wherein the hypomineralised dental surface or subsurface, or lesion, is contacted with the liquid composition in (i) for at least about a few seconds to at about 5 minutes.

69. A method according to claim 68, wherein the hypomineralised dental surface or subsurface, or lesion, is contacted with the liquid composition in (i) for at least about 5 minutes to about 20 minutes.

70. A method according to any one of claims 24 to 69, wherein the method further comprises acid etching the hypomineralised surface, subsurface or lesion, prior to contacting with the liquid composition in (i).

71. A method according to any one of claims 24 to 70, wherein the phosphopeptide is a casein phosphopeptide.

72. A method according to any one of claims 1 to 71, wherein the liquid, further and/or mixed compositions are applied to the dental surface, subsurface or lesion by a dental health care professional.

73. A method according to claim 72, wherein the liquid, further and/or mixed compositions are applied to the dental surface, subsurface or lesion using a microbrush.

74. A method according to any one of claims 1 to 73, wherein the method further comprises a step of identifying a subject having a white spot lesion, a fluorotic lesion, a caries lesion, or a lesion caused by tooth erosion.

75. A cosmetic method of reducing visibility of a hypomineralised dental surface or subsurface, the method comprising:
(i) contacting the hypomineralised dental surface or subsurface with a liquid composition comprising at least 40% w/v of phosphopeptide (PP)-stabilized amorphous calcium phosphate (ACP) and/or amorphous calcium fluoride phosphate (ACFP) at a pH of less than or equal to pH 6; and
(ii) subsequently to (i), raising the pH of the liquid composition applied to the hypomineralised dental surface or subsurface to equal to, or greater than, about 9, thereby forming a gel in and/or on the hypomineralised dental surface or subsurface,
thereby reducing visibility of a hypomineralised dental surface or subsurface.

76. A cosmetic method reducing visibility of a hypomineralised dental surface or subsurface, the method comprising contacting the hypomineralised dental surface or subsurface with a liquid composition comprising at least 40% w/w of phosphopeptide (PP)-stabilized amorphous calcium phosphate (ACP) and/or amorphous calcium fluoride phosphate (ACFP) at a pH of greater than or equal to pH 6 but less than or equal to pH 8,
thereby reducing visibility of a hypomineralised dental surface or subsurface.

77. A kit for reducing visibility of a hypomineralised dental surface or subsurface, the kit comprising or consisting of: (a) a liquid composition comprising at least 40% by weight of phosphopeptide (PP)-stabilized amorphous calcium phosphate (ACP) and/or amorphous calcium fluoride phosphate (ACFP) at a pH of less than or equal to pH 6, and (b) a further composition of alkaline pH.

78. A kit according to claim 77, wherein the kit further comprises written instructions for use in any method according to claims 1 to 76.

79. A kit for reducing visibility of a hypomineralised dental surface or subsurface comprising or consisting of:
(a) a first composition comprising a powder of phosphopeptide stabilized ACP and/or ACFP;
(b) a second composition comprising a solution of fluoride at a pH of less than or equal to pH 6; and
(c) a third composition of alkaline pH.

80. A kit according to any one of claims 77 to 79, wherein the alkaline pH of the third composition is a pH of about 9, 10, 11, 12, 13 or 14.

81. A kit according to claim 77 or 78, wherein the first composition comprises an amount of phosphopeptide stabilized ACP and/or ACFP that when mixed with the second composition, a liquid composition comprising at least 40% w/v of phosphopeptide stabilized ACP and/or ACFP is formed.

82. A kit for reducing visibility of a hypomineralised dental surface or subsurface, the kit comprising or consisting of:
(a) 5 g of CPP-ACP and/or CPP-ACFP,
(b) 5 ml of 0.73 M NaF in 1.146 M HCl, and
(c) 1.5 M NaOH.

83. A kit according to claim 82, wherein the kit further includes two microbrushes.

84. A method or process for preparing a liquid composition comprising at least 40% w/v PP stabilized ACP and/or ACFP, the method or process comprising or consisting of:
mixing a solvent and a powder comprising or consisting of PP stabilized-ACP and/or ACFP, and
maintaining the pH below 7.

85. A method or process for preparing a liquid composition comprising at least 40% w/v PP stabilized ACP and/or ACFP, the method or process comprising or consisting of:
mixing a solvent to a powder comprising or consisting of PP stabilized ACP and/or ACFP, and
lowering the pH below 7, preferably, the pH is lowered to, or below, 6, preferably 5.5.

86. A method or process according to claim 84 or 85, wherein the pH is maintained at, or below, 6, preferably the pH is maintained at, or below, 5.5.

87. A method or process according to any one of claims 84 to 86, further comprising the following steps to prepare a powder comprising or consisting of PP stabilized-ACP and/or ACFP:
admixing one or more solutions comprising phosphopeptides, calcium ions, phosphate ions, hydroxide ions and optionally fluoride ions, while maintaining the pH at about 7.0 or above, preferably about 9, to form a solution comprising stabilized-ACP and/or ACFP, and
drying the solution comprising PP stabilized-ACP and/or ACFP,
thereby forming a powder comprising or consisting of PP stabilized-ACP and/or ACFP.

88. A method or process according to claim 87, wherein the drying is spray drying or freeze drying.

89. A method or process according to claim 87 or 88, further comprising the step of filtering the solution comprising PP stabilized-ACP and/or ACFP, prior to drying, to form a retentate, wherein the retentate is subsequently dried to form powder comprising or consisting of PP stabilized-ACP and/or ACFP.
